(19) United States
(12) Patent Application Publication
(10) Pub. No.: US 2006/0287282 A1
(43) Pub. Date: Dec. 21, 2006

(54) Compositions comprising a SARM ad GnRH agonist or a GnRH antagonist, and methods of use thereof

(76) Inventors: Mitchell S. Steiner, Germantown, TN (US); Karen A. Veverka, Cordova, TN (US)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/003,938

(22) Filed: Dec. 6, 2004

Related U.S. Application Data
(63) Continuation-in-part of application No. 10/270,232, filed on Oct. 15, 2002, now Pat. No. 6,838,484.
Application No. 10/270,232 is a continuation-in-part of application No. 09/935,044, filed on Aug. 23, 2001, now Pat. No. 6,492,554.
Application No. 10/270,232 is a continuation-in-part of application No. 09/935,045, filed on Aug. 23, 2001, now Pat. No. 6,569,896.
(60) Provisional application No. 60/300,083, filed on Jun. 25, 2001.

(51) Int. Cl. A61K 31/675; A61K 31/555; A61K 31/277; A61K 31/32; A61K 31/165

(52) U.S. Cl. 514/114; 514/617; 514/618; 514/620; 514/493; 514/522; 514/616; 514/599; 514/357; 514/400; 514/184

(57) ABSTRACT

The present invention relates to compositions comprising a selective androgen receptor modulators (SARM) and a gonadotropin releasing hormone (GnRH) agonist or a GnRH antagonist, and their use, inter-alia for treating hormone-associated conditions in males and females, which arise as a result of androgen decline, suppression or abrogation, or in treating, suppressing, inhibiting or preventing prostate cancer.

### CROSS REFERENCE TO RELATED APPLICATIONS

This Application is a Continuation-in-Part Application of U.S. Ser. No. 10/270,232, filed Oct. 15, 2002, which is a Continuation-in-Part Application of U.S. Ser. No. 09/935,044, filed Aug. 23, 2001 and of U.S. Ser. No. 09/935,045, filed Aug. 23, 2001, which are Continuation-in-Part Applications of U.S. Ser. No. 09/644,970 filed Aug. 24, 2000; and claims priority of U.S. Ser. No. 60/300,083, filed Jun. 25, 2001, which are hereby incorporated by reference.

### FIELD OF INVENTION

The present invention relates to compositions comprising a selective androgen receptor modulator (SARM) and a gonadotropin releasing hormone (GnRH) agonist or antagonist. The combinations are useful for a) treating, preventing, suppressing, inhibiting or reducing the incidence of a condition associated with androgen decline in a male subject, such as: sexual dysfunction, decreased sexual libido, erectile dysfunction, hypogonadism, sarcopenia, osteopenia, osteoporosis, alterations in cognition and mood, depression, anemia, hair loss, obesity or muscle wasting; b) treating, preventing, suppressing, inhibiting, reducing the incidence of, or reducing relapse of prostate cancer in a male subject; c) treating, preventing, suppressing, inhibiting, or reducing the incidence of precancerous precursors of prostate carcionogenesis in a male subject; or d) treating, preventing, suppressing, inhibiting or reducing the incidence of a hormone-associated disorder in a female subject, such as: sexual dysfunction, decreased sexual libido, hypogonadism, sarcopenia, osteopenia, osteoporosis, alterations in cognition and mood, depression, anemia, hair loss, menstrual disorders, infertility, endometriosis, polycystic ovarian syndome, hirsutism, acne, dyslipidemia, hypertension, obesity, muscle wasting, uterine, breast or ovarian cancer.

### BACKGROUND OF THE INVENTION

The androgen receptor (“AR”) is a ligand-activated transcriptional regulatory protein that mediates induction of male sexual development and function through its activity with endogenous androgens. Androgens are generally known as the male sex hormones. The androgenic hormones are steroids, which are produced in the body by the testes and the cortex of the adrenal gland or can be synthesized in the laboratory. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Testosterone is the principal steroid secreted by the testes and is the primary circulating androgen found in the plasma of males. Testosterone is converted to DHT by the enzyme 5 alpha-reductase in many peripheral tissues. DHT is thus thought to serve as the intracellular mediator for most androgen actions (Zhou, et al., Molec. Endocrinol. 9:208-18 (1995)). Other steroidal androgens include esters of testosterone, such as the cypionate, propionate, phenylpropionate, cyclopentylpiopionate, isocarporate, enanthate, and decanoate esters, and other synthetic androgens such as 7-Methyl-Nortestosterone (“MENT”) and its acetate ester (Sundaram et al., “7 Alpha-Methyl-Nortestosterone(MENT): The Optimal Androgen For Male Contraception,” Ann. Med., 25:199-205 (1993) (“Sundaram”)).

It has been found that androgen suppression or abrogation may result in beneficial effects, in a number of diseases and clinical applications, in males and in females.

Prostate cancer is one of the most frequently occurring cancers among men in the United States, with hundreds of thousands of new cases diagnosed each year. Unfortunately, over sixty percent of newly diagnosed cases of prostate cancer are found to be pathologically advanced, with no cure and a dismal prognosis. One approach to this problem is to find prostate cancer earlier through screening programs and thereby reduce the number of advanced prostate cancer patients. Another strategy, however, is to develop drugs to prevent prostate cancer. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer. Thus, the development of treatment and preventative strategies against prostate cancer may have the greatest overall impact both medically and economically against prostate cancer. Androgen suppression therapy has been shown to be beneficial in treating prostate cancer.

Androgen inhibition, abrogation, and decline, however is associated with a number of undesirable effects. Androgen decline in the aging male (ADAM), for example, is a progressive decrease in androgen production, which is common in males after middle age. The syndrome is characterized by alterations in the physical and intellectual domains that correlate with and can be corrected by manipulation of the androgen milieu.

ADAM is characterized biochemically by a decrease not only in serum androgen, but also in other hormones, such as growth hormone, melatonin and dehydroepiandrosterone. Clinical manifestations include fatigue, depression, decreased libido, sexual dysfunction, erectile dysfunction, sarcopenia, osteopenia, osteoporosis, benign prostate hyperplasia, hypogonadism, alterations in mood and cognition, depression, anemia, obesity, hair loss and prostate cancer.

The onset of ADAM is unpredictable and its manifestations are subtle and variable, which has led to a paucity of interest in its diagnosis, monitoring and treatment. Innovative approaches are urgently needed at both the basic science and clinical levels to treat ADAM, and symptoms associated with androgen suppression or abrogation.

In women, androgen suppression or abrogation, has been shown therapeutic in clinical settings, such as, for example, in treating Polycystic Ovarian Syndrome (PCOS), endometriosis, hirsutism and and other hormone-associated disorders. As is the case in male subjects, however, androgen suppression, abrogation or decline is accompanied by a variety of undesirable clinical conditions and symptoms.

Androgen Deficiency in Female (ADIF) refers to a variety of hormone-related conditions including, common in females after middle agest. The syndrome is characterized by sexual dysfunction, decreased sexual libido, hypogonadism, sarcopenia, osteopenia, osteoporosis, alterations in cognition and mood, anemia, depression, anemia, hair loss, obesity, endometriosis, breast cancer, uterine cancer and ovarian cancer.

New innovative approaches are urgently needed at both the basic science and clinical levels to develop compositions and treatment regimens, which provide the beneficial effects of androgen suppression or abrogation, without deleterious side-effects as a result of the treatment.

### SUMMARY OF THE INVENTION

In one embodiment, this invention provides a composition comprising a gonadotropin releasing hormone agonist, its analog, isomer, metabolite, derivative, pharmaceutically acceptable salt, N-oxide, hydrate, acetate or any combination thereof and an androgen receptor targeting agent (ARTA), which is a selective androgen receptor modulator (SARM). In one embodiment, the SARM compounds bind irreversibly to the androgen receptor. In another embodiment, the SARM compounds are androgen receptor antagonists, which bind irreversibly to the androgen receptor. In another embodiment, the SARM compounds are allcylating agents.

In another embodiment, this invention provides a composition comprising a gonadotropin releasing hormone antagonist, its analog, isomer, metabolite, derivative, pharmaceutically acceptable salt, N-oxide, hydrate, acetate or any combination thereof and an androgen receptor targeting agent (ARTA), which is a selective androgen receptor modulator (SARM). In one embodiment, the SARM compounds bind irreversibly to the androgen receptor. In another embodiment, the SARM compounds are androgen receptor antagonists, which bind irreversibly to the androgen receptor. In another embodiment, the SARM compounds are alkylating agents.

The compositions are useful, in some embodiments, for a) treating, preventing, suppressing, inhibiting or reducing the incidence of a condition associated with androgen decline in a male subject, such as: sexual dysfunction, decreased sexual libido, erectile dysfunction, hypogonadism, sarcopenia, osteopenia, osteoporosis, alterations in cognition and mood, depression, anemia, hair loss, obesity or muscle wasting; b) treating, preventing, suppressing, inhibiting, reducing the incidence of, or reducing relapse of prostate cancer in a male subject; c) treating, preventing, suppressing, inhibiting, or reducing the incidence of precancerous precursors of prostate carcionogenesis in a male subject; or d) treating, preventing, suppressing, inhibiting or reducing the incidence of a hormone-associated disorder in a female subject, such as: sexual dysfunction, decreased sexual libido, hypogonadism, sarcopenia, osteopenia, osteoporosis, alterations in cognition and mood, depression, anemia, hair loss, menstrual disorders, infertility, endometriosis, polycystic ovarian syndome, hirsutism, acne, dyslipidemia, hypertension, obesity, muscle wasting, uterine, breast or ovarian cancer.

In one embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula I: 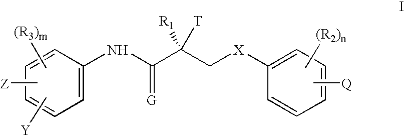
x is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalcyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 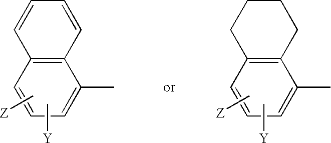
Z is NO₂, CN, COR, COOH, or CONHR;
Y is CF₃ F, Br, Cl, I, CN, or SnR₃,
Q is SCN, NCS, OCN, or NCO;
n is an integer of 1-4;
m is an integer of 1-3; and
wherein all unspecified positions can be substituted or unsubstituted.

In one embodiment, G in the compound of formula I is O. In another embodiment, X in the compound of formula I is O. In another embodiment, T in the compound of formula I is OH. In another embodiment, R₁ the compound of formula I is CH₃. In another embodiment, Z the compound of formula I is NO₂. In another embodiment, Z in the compound of formula I is CN. In another embodiment, Y in the compound of formula I is CF₃. In another embodiment, Q in the compound of formula I is NCS. In another embodiment, Q in the compound of formula I is in the para position. In another embodiment, Z in the compound of formula I is in the para position. In another embodiment, Y in the compound of formula I is in the meta position. In another embodiment, G in the compound of formula I is O, T is OH, R₁ is CH₃, X is O, Z is NO₂, Y is CF₃, and Q is NCS.

According to this aspect of the invention, and in another embodiment, the composition will comprise an analog, derivative, isomer, metabolite, pharmaceutically acceptable salt, pharmaceutical product, hydrate or N-oxide of the SARM compound of formula I, or any combination thereof.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula II:

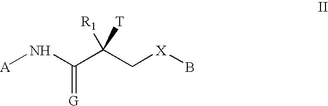

wherein X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalcyl, CH₂F, CHF₂, CF₃, CF₂CF₃, aryl, phenyl, halogen, alkenyl or OH;
A is a ring selected from: 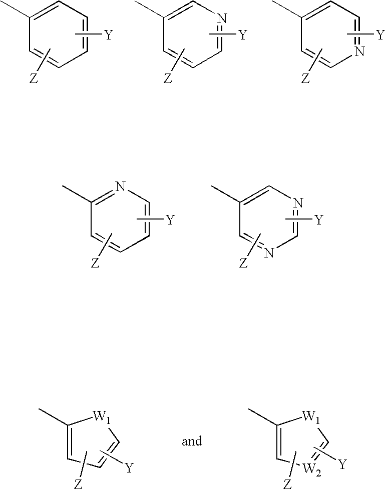
B is a ring selected from:

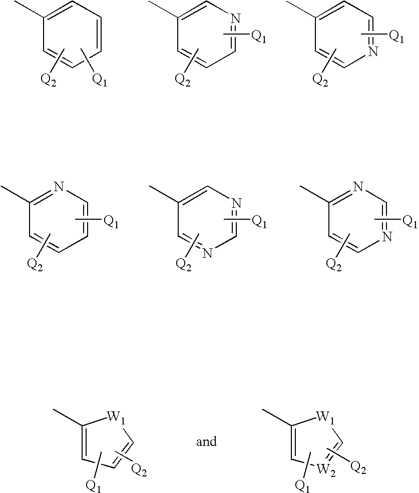

wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 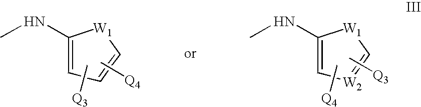
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO; and
wherein all unspecified positions can be substituted or unsubstituted.

In one embodiment, G the compound of formula II is O. In another embodiment, X in the compound of formula II is O. In another embodiment, T in the compound of formula II is OH. In another embodiment, R₁ in the compound of formula II is CH₃ In another embodiment, Z in the compound of formula II is NO₂. In another embodiment, Z in the compound of formula II is CN. In another embodiment, Y in the compound of formula II is CF₃. In another embodiment, Q₁ in the compound of formula II is NCS. In another embodiment, Q₁ in the compound of formula II is in the para position. In another embodiment, Z in the compound of formula II is in the para position. In another embodiment, Y in the compound of formula II is in the meta position. In another embodiment, G in the compound of formula II is O, T is OH, R₁ is CH₃, X is O, Z is NO₂, Y is CF₃, and Q₁ is NCS.

According to this aspect of the invention, and in another embodiment, the composition will comprise an analog, derivative, isomer, metabolite, pharmaceutically acceptable salt, pharmaceutical product, hydrate or N-oxide of the SARM compound of formula II, or any combination thereof.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula III:

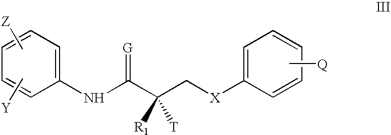

wherein X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment, G in the compound of formula III is O. In another embodiment, X in the compound of formula III is O. In another embodiment, T in the compound of formula III is OH. In another embodiment, R₁ in the compound of formula III is CH₃. In another embodiment, Z in the compound of formula III is NO₂ In another embodiment, Z in the compound of formula III is CN. In another embodiment, Y in the compound of formula III is CF₃. In another embodiment, Q in the compound of formula III is NCS. In another embodiment, Q in the compound of formula III is in the para position. In another embodiment, Z in the compound of formula III is in the para position. In another embodiment, Y in the compound of formula III is in the meta position. In another embodiment, G in the compound of formula III is O, T is OH, R₁ is CH₃, X is O, Z is NO₂, Y is CF₃, and Q is NCS.

According to this aspect of the invention, and in another embodiment, the composition will comprise an analog, derivative, isomer, metabolite, pharmaceutically acceptable salt, pharmaceutical product, hydrate or N-oxide of the SARM compound of formula III, or any combination thereof.

The substituent R is defined herein as an alkyl, haloalkyl, dihaloalkyl, trihaloalkyl, CH2F, CHF2, CF3, CF2CF3; aryl, phenyl, F, Cl, Br, I, alkenyl, or hydroxyl (OH).

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula IV:

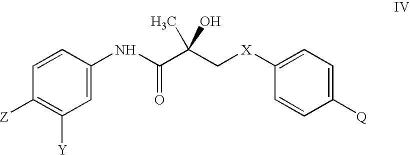

wherein X is a bond, O, CH₂, NH, S, Se, PR, NO or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH.

According to this aspect of the invention, and in another embodiment, the composition will comprise an analog, derivative, isomer, metabolite, pharmaceutically acceptable salt, pharmaceutical product, hydrate or N-oxide of the SARM compound of formula IV, or any combination thereof

In one embodiment, X in the compound of formula IV is O. In another embodiment, Z in the compound of formula IV is NO₂. In another embodiment, Z in the compound of formula IV is CN. In another embodiment, Y in the compound of formula IV is CF₃. In another embodiment, Q in the compound of formula IV is NCS.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula V, and/or an analog, derivative, isomer, metabolite, pharmaceutically acceptable salt, pharmaceutical product, hydrate, N-oxide, or any combination thereof.

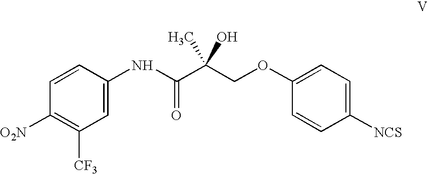

In one embodiment, the SARM compound of any of formulas I-V is an androgen receptor antagonist. In another embodiment, the SARM compound of any of formulas I-V binds irreversibly to an androgen receptor. In another embodiment, the SARM compound of any of formulas I-V is an androgen receptor antagonist, which binds irreversibly to an androgen receptor. In another embodiment, the SARM compound of any of formulas I-V is an alkylating agent.

In another embodiment, the present invention provides a method of treating, preventing, suppressing, inhibiting or reducing the incidence of a condition associated with androgen decline in a male subject, wherein said condition is sexual dysfunction, decreased sexual libido, erectile dysfunction, hypogonadism, sarcopenia, osteopenia, osteoporosis, osteoporosis, alterations in cognition and mood, depression, anemia, hair loss, obesity or muscle wasting said method comprising the step of administering to said subject a composition of this invention.

In another embodiment, the present invention provides a method of treating, preventing, suppressing, inhibiting or reducing the incidence of a condition associated with androgen decline in a male subject, wherein said condition is sexual dysfunction, decreased sexual libido, erectile dysfunction, hypogonadism, sarcopenia, osteopenia, osteoporosis, alterations in cognition and mood, depression, anemia, hair loss, obesity or muscle wasting, said method comprising the step of administering to said subject a selective androgen receptor modulator (SARM) and a gonadotropin releasing hormone (GnRH) agonist or antagonist.

In another embodiment, the present invention provides a method of treating, preventing, suppressing, inhibiting, reducing the incidence of, or reducing relapse of prostate cancer in a male subject, said method comprising the step of administering to said subject a composition of this invention.

In another embodiment, the present invention provides a method of treating, preventing, suppressing, inhibiting, reducing the incidence of, or reducing relapse of prostate cancer in a male subject, said method comprising the step of administering to said subject a selective androgen receptor modulator (SARM) and a gonadotropin releasing hormone (GnRH) agonist or antagonist.

In another embodiment, this invention provides a method of treating, preventing, suppressing, inhibiting, or reducing the incidence of precancerous precursors of prostate carcionogenesis in a male subject, said method comprising the step of administering to said subject a composition of this invention.

In another embodiment, this invention provides a method of treating, preventing, suppressing, inhibiting or reducing the incidence of precancerous precursors of prostate cancer in a male subject, said method comprising the step of administering to said subject a selective androgen receptor modulator (SARM) and a gonadotropin releasing hormone (GnRH) agonist or antagonist.

In another embodiment, this invention provides a method of treating, preventing, suppressing, inhibiting or reducing the incidence of a condition associated with androgen decline in a female subject, wherein said condition is sexual dysfunction, decreased sexual libido, hypogonadism, sarcopenia, osteopenia, osteoporosis, alterations in cognition and mood, depression, anemia, hair loss, obesity or muscle wasting, said method comprising the step of administering to said subject to said subject a composition of this invention.

In another embodiment, this invention provides a method of treating, preventing, suppressing, inhibiting or reducing the incidence of a hormone-associated disorder in a female subject, wherein said condition is sexual dysfunction, decreased sexual libido, hypogonadism, sarcopenia, osteopenia, osteoporosis, alterations in cognition and mood, depression, anemia, hair loss, menstrual disorders, infertility, endometriosis, polycystic ovarian syndome, hirsutism, acne, dyslipidemia, hypertension, obesity, muscle wasting, uterine, breast or ovarian cancer, said method comprising the step of administering to said subject a selective androgen receptor modulator (SARM) and a gonadotropin releasing hormone (GnRH) agonist or antagonist.

In one embodiment, a selective androgen receptor modulator (SARM) is administered concurrently with a gonadotiopin releasing hormone (GnRH) agonist or antagonist, according to a method of this invention. In another embodiment, a selective androgen receptor modulator (SARM) is administered prior to adminstering a gonadotropin releasing hormone (GnRH) agonist or antagonist, according to a method of this invention. In another embodiment, a selective androgen receptor modulator (SARM) is administered following adminstration of a gonadotropin releasing hormone (GnRH) agonist or antagonist, according to a method of this invention.

### DETAILED DESCRIPTION OF THE INVENTION

In one embodiment, this invention provides a composition comprising a gonadotropin releasing hormone agonist, its analog, isomer, metabolite, derivative, pharmaceutically acceptable salt, N-oxide, hydrate, acetate or any combination thereof and an androgen receptor targeting agent (ARTA), which is a selective androgen receptor modulator (SARM). In one embodiment, the SARM compounds bind irreversibly to the androgen receptor. In another embodiment, the SARM compounds are androgen receptor antagonists, which bind irreversibly to the androgen receptor. In another embodiment, the SARM compounds are alkylating agents.

In some embodiments of this invention, the compositions of this invention are useful, inter-alia, for a) treating, preventing, suppressing, inhibiting or reducing the incidence of a condition associated with androgen decline in a male subject, such as: sexual dysfunction, decreased sexual libido, erectile dysfunction, hypogonadism, sarcopenia, osteopenia, osteoporosis, alterations in cognition and mood, depression, anemia, hair loss, obesity or muscle wasting; b) treating, preventing, suppressing, inhibiting, reducing the incidence of, or reducing relapse of prostate cancer in a male subject; c) treating, preventing, suppressing, inhibiting, or reducing the incidence of precancerous precursors of prostate carcionogenesis in a male subject; or d) treating, preventing, suppressing, inhibiting or reducing the incidence of a hormone-associated disorder in a female subject, such as: sexual dysfunction, decreased sexual libido, hypogonadism, sarcopenia, osteopenia, osteoporosis, alterations in cognition and mood, depression, anemia, hair loss, menstrual disorders, infertility, endometriosis, polycystic ovarian syndome, hirsutism, acne, dyslipidemia, hypertension, obesity, muscle wasting, uterine, breast or ovarian cancer.

In other embodiments, compositions of this invention are useful, inter-alia, fore) male contraception; f) treatment of diseases associated with polycystic ovarian syndromes and syndrome; or g) preventing and/or treating dry eye conditions.

In one embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula I: 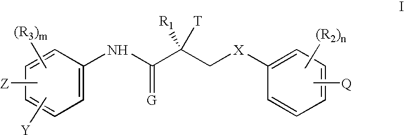
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 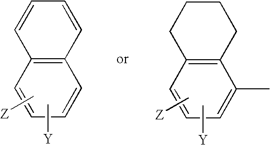
Z is NO₂, CN, COR, COOH, or CONHR,
Y is CF₃, F, Br, Cl, I, CN, or SnR₃;
Q is SCN, NCS, OCN, or NCO
n is an integer of 1-4;
m is an integer of 1-3; and
wherein all unspecified positions can be substituted or unsubstituted.

In one embodiment, G in the compound of formula I is O. In another embodiment, X in the compound of formula I is O. In another embodiment, T in the compound of formula I is OH. In another embodiment, R₁ the compound of formula I is CH₃. In another embodiment, Z the compound of formula I is NO₂. In another embodiment, Z in the compound of formula I is CN. In another embodiment, Y in the compound of formula I is CF₃. In another embodiment, Q in the compound of formula I is NCS. In another embodiment, Q in the compound of formula I is in the para position. In another embodiment, Z in the compound of formula I is in the para position. In another embodiment, Y in the compound of formula I is in the meta position. In another embodiment, G in the compound of formula I is O, T is OH, R₁ is CH₃, X is O, Z is NO₂, Y is CF₃, and Q is NCS.

According to this aspect of the invention, and in another embodiment, the composition will comprise an analog, derivative, isomer, metabolite, pharmaceutically acceptable salt, pharmaceutical product, hydrate or N-oxide of the SARM compound of formula I, or any combination thereof.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula II:

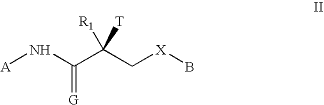

wherein X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 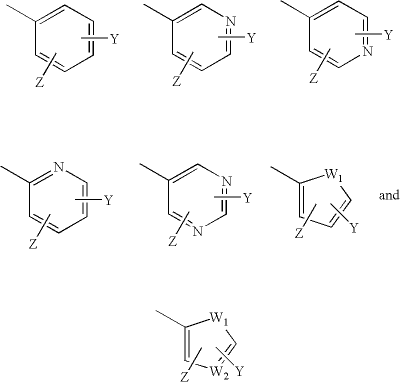
B is a ring selected from:

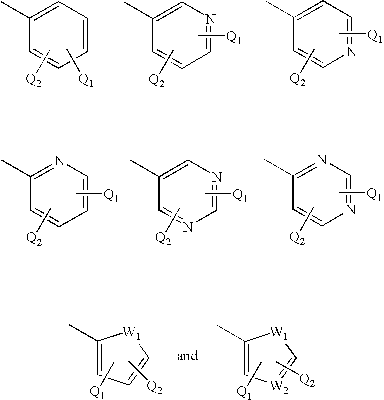

wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 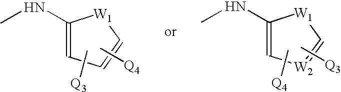
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO; and
wherein all unspecified positions can be substituted or unsubstituted

In one embodiment, G the compound of formula II is O. In another embodiment, X in the compound of formula II is O. In another embodiment, T in the compound of formula II is OH. In another embodiment, R₁ in the compound of formula II is CH₃. In another embodiment, Z in the compound of formula II is NO₂ In another embodiment, Z in the compound of formula II is CN. In another embodiment, Y in the compound of formula II is CF₃. In another embodiment Q₁ in the compound of formula II is NCS. In another embodiment, Q₁ in the compound of formula II is in the para position. In another embodiment, Z in the compound of formula II is in the para position. In another embodiment, Y in the compound of formula II is in the meta position. In another embodiment, G in the compound of formula II is O, T is OH, R₁ is CH₃, X is O, Z is NO₂, Y is CF₃, and Q₁ is NCS.

According to this aspect of the invention, and in another embodiment the composition will comprise an analog, derivative, isomer, metabolite, pharmaceutically acceptable salt, pharmaceutical product, hydrate or N-oxide of the SARM compound of formula II, or any combination thereof.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula III:

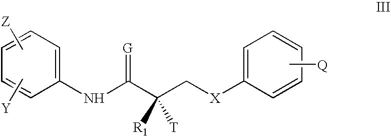

wherein X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment, G in the compound of formula III is O. In another embodiment, X in the compound of formula III is O. In another embodiment, T in the compound of formula III is OH. In another embodiment, R₁ in the compound of formula III is CH₃. In another embodiment, Z in the compound of formula III is NO₂. In another embodiment, Z in the compound of formula III is CN. In another embodiment, Y in the compound of formula III is CF₃. In another embodiment, Q in the compound of formula III is NCS. In another embodiment, Q in the compound of formula III is in the para position. In another embodiment, Z in the compound of formula III is in the para position. In another embodiment, Y in the compound of formula III is in the meta position. In another embodiment, G in the compound of formula III is O, T is OH, R₁ is CH₃, X is O, Z is NO₂, Y is CF₃, and Q is NCS.

According to this aspect of the invention, and in another embodiment, the composition will comprise an analog, derivative, isomer, metabolite, pharmaceutically acceptable salt, pharmaceutical product, hydrate or N-oxide of the SARM compound of formula III, or any combination thereof.

The substituent R is defined herein as an alkyl, haloalkyl, dihaloalkyl, trihaloalkyl, CH2F, CHF2, CF3, CF2CF3; aryl, phenyl, F, Cl, Br, I, alkenyl, or hydroxyl (OH).

In one embodiment, the term “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons In another embodiment, the alkyl group has 1-6 carbons. In another embodiment, the alkyl group has 1-4 carbons. The alkyl group may, in other embodiments be unsubstituted or substituted by one or more groups selected from halogen (e.g. F, Cl, Br, I), hydroxy, alkoxy carbonyl, amido, alkylamido, dialkylamino, nitro, amino, alkylamino, dialkylamino, carboxyl, thio and thioalkyl.

In one embodiment, the term “haloalkyl” refers to an alkyl group as defined above, which is substituted by one or more halogen atoms, e.g. by F, Cl, Br or I. In another embodiment, the term “halogen” refers to elements of Group VII or the periodic table, e.g. F, Cl, Br or I.

In another embodiment, the term “aryl” group refers to an aromatic group having at least one carbocyclic aromatic group or heterocyclic aromatic group, which may be unsubstituted or substituted by one or more groups selected from halogen (e.g. F, Cl, Br, I), haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

In another embodiment, the term “hydroxyl” group refers to an OH group. An “alkenyl” group refers to a group having at least one carbon to carbon double bond.

In another embodiment, the term “arylalkyl” group refers to an alkyl bound to an aryl, wherein alkyl and aryl are as defined above. An example of an aralkyl group is a benzyl group.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula IV:

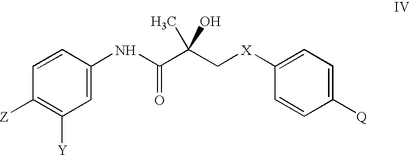

wherein X is a bond, O, CH₂, NH, S, Se, PR, NO or NR;
Z is NO₂, CN, COOH, COR, NHCOR or CONHR;
Y is CF₃, F, I, Br, Cl, CN, CR, or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH.

According to this aspect of the invention, and in another embodiment, the composition will comprise an analog, derivative, isomer, metabolite, pharmaceutically acceptable salt, pharmaceutical product, hydrate or N-oxide of the SARM compound of formula IV, or any combination thereof.

In one embodiment, X in the compound of formula IV is O. In another embodiment, Z in the compound of formula IV is NO₂. In another embodiment, Z in the compound of formula IV is CN. In another embodiment, Y in the compound of formula IV is CF₃. In another embodiment, Q in the compound of formula IV is NCS.

In another embodiment, the present invention provides a composition comprising a gonadotropin releasing hormone agonist or antagonist, its analog, isomer, metabolite, derivative, pharmaceutically acceptable salt, N-oxide, hydrate, acetate or any combination thereof and a selective androgen receptor modulator (SARM) compound represented by the structure of formula V, and/or an analog, derivative, isomer, metabolite, pharmaceutically acceptable salt, pharmaceutical product, hydrate, N-oxide, or any combination thereof.

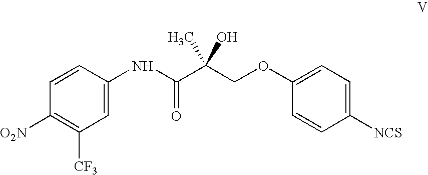

In one embodiment, the SARM compound of any of formulas I-V is an androgen receptor antagonist. In another embodiment, the SARM compound of any of formulas I-V binds irreversibly to an androgen receptor. In another embodiment, the SARM compound of any of formulas I-V is an androgen receptor antagonist, which binds irreversibly to an androgen receptor. In another embodiment, the SARM compound of any of formulas I-V is an allcylating agent.

In one embodiment, the composition comprises a SARM and a GnRH agonist. In one embodiment, the terms “GnRH agonist” and “LHRH agonist”, are used interchangeably, and may refer, in another embodiment, to a compound that stimulates the luteinizing hormone releasing hormone receptor such that luteinizing hormone is released (e.g., a compound that mimics the activity of LHRH). An LHRH agonist can have greater LH-releasing activity than natural LHRH, in one embodiment. Many such LHRH agonists are known in the art. Commercially available LHRH agonists may be used in the compositions and methods of this invention, and may include leuprolide (trade name: Lupron®; Abbott/TAP), decapeptyl (trade name: Debiopharm®; Ipsen/Beaufour), lutrelin (Wyeth), cystorelin (Hoechst), leuprorelin (leuprorelin acetate), Goserelin (U.S. Pat. No. 4,100,274 A, JP 52-136172 A), Buserelin (U.S. Pat. No. 4,024,248, German Patent No.2438352, JP 5141359 A), Triptorelin (U.S. Pat. No. 4,010,125 A, JP 52-31073 A), Nafarelin (U.S. Pat. No. 4,234,571 A, JP 55-164663 A, JP 63-264498 A, JP 64-25794 A), Histrelin, Deslorelin (U.S. Pat. No. 4,569,967 A, U.S. Pat. No. 4,218,439 A), Meterelin (PCT WO 91/18016), Gonadrelin (German Patent No.2213737), Avorelin, compound PTL 03001 (5-oxo-L-propyl-L-histidyl-L-tryptophyl-L-seryl-L-tyrosyl-D-tryptophyl-L-leucyl-L-arginyl-N-ethyl-L-prolinamide) (Peptech), compound AN 207 (6-[N6-[5-[2-[1,2,3,4,6,11-hexahydro-2,5,12-trihydroxy-7-mehoxy-6,11 -dioxo-4-[[2,3,6-trideoxy-3-(2,3-dihydro-1H-pyrrol-1-yl).alpha.-L-lyxo-hexopyranosyl]oxy]-2-naphthacenyl]-1,5-dioxopentyl]-D-lysine]-, (2S-cis)-) (ASTA Medica Inc.), compound AN 238 L-threoninamide, N-[5-[2-[(2S,4S)-1,2,3,4,6,11-hexahydro-2,5,12-trihydroxy-7-methoxy-6,11-dioxo-4-[[2,3,6-trideoxy-3-(2,3-dihydro-1H-pyrrl-1-yl).alpha.-L-lyxo-hexopyianosyl]oxy]-2-naphthacenyl]-2-oxoetllioxy]-1,5-dioxopentyl)-D-phenylalanyl-L,-cysteinyl-L-tosyl-D-tryptophyl-L-lysyl-L-valyl-L-cysteinyl-cyclic (2.fwdarw.7)-disulfide (ASTA Medica Inc.), compound SPD 424 (LHRH-hydrogel implant) (Shire Pharmaceuticals Group), or a pharmaceutically acceptable salt thereof, or any combination thereof.

In another embodiment, the GnRH agonist will be characterized by the formula: 5-oxo-Pro-His-Trp-Ser-Tyr-DLeu-Leu-Arg-Pro-NH-C2H5 or its salt

In another embodiment, the compositions of this invention comprise a SARM and a GnRH antagonist. In one embodiment, the term “GnRH antagonist” and “LHRH antagonist” are used interchangeably, and refer, in another embodiment, to a compound that inhibits the luteinizing hormone releasing hormone receptor such that release of luteinizing hormone is inhibited

In one embodiment, the GnRH antagonists used in compositions and methods of this invention may include GnRH antagonists that have been described in the art such as cetrorelix and Nal-Glu; including antagonists described in e.g., U.S. Pat. No. 5,470,947 to Follcers et al.; Folkers et al., PCT Publication No. WO 89/01944; U.S. Pat. No. 5,413,990 to Haviv; U.S. Pat. No. 5,300,492 to Haviv; U.S. Pat No. 5,371,070 to Koerber et al., U.S. Pat. No. 5,296,468 to Hoeger et al.; U.S. Pat. No. 5,171,835 to Janaky et al.; U.S. Pat No. 5,003,011 to Coy et al; U.S. Pat. No. 4,431,635 to Coy; U.S. Pat. No. 4,992,421 to De et al.; U.S. Pat. No. 4,851,385 to Roeske; U.S. Pat. No. 4,801,577 to Nestor, Jr. et al.; and U.S. Pat. No. 4,689,396 to Roeske et al., abarelix, ramorelix, teverelix, ganirelix, or any combination thereof.

In another embodiment, the gonadotropin releasing hormone antagonist has low histamine-releasing activity (e.g., an ED50 for histamine release in a standard in vitro histamine release assay of at least 3 μg/ml, more preferably at least 5 μg/ml, and still more preferably at least 10 μg/ml) and is water-soluble.

In another embodiment, the gonadotropin releasing hormone antagonist is a peptide, characterized by the structure: Ac-D-Nal-4-Cl-Phe-D-Pal-Ser-Tyr-D-Pal(N—O)-Leu-Lys(iPr)-Pro-D-Ala-NH₂, or in another embodiment comprising a structure of Ac-D-Nal-4-Cl-D-Phe-D-Pal-Ser-Tyr-D-Pal(CH2-COO-)-Leu-Lys(iPr)-Pro-Ala-NH₂, or in another embodiment, Ac-Sar-4-Cl-D-Phe-D-Nal-Ser-Tyr-D-Pal(Bzl)-Leu-Lys(iPr)-Pro-Ala-NH₂ or a pharmaceutically acceptable salt thereof.

The compositions of this invention will have effective amounts of the GnRH agonist or antagonist and SARM together with suitable diluents, preservatives, solubilizers, emulsifiers, adjuvants, and/or carriers. An “effective amount” refers, in one embodiment, to that amount which provides a desired effect for a given application, as described further hereinunder, and in another embodiment, may be a function of administration regimen. Such compositions may, in other embodiments, be liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions may, in other embodiments, influence the physical state, solubility, stability, rate of in vivo release, and rate of in vivo clearance. Controlled or sustained release compositions are envisioned in the instant invention, and may include, in other embodiments, formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral. In one embodiment, the pharmaceutical composition is formulated for administration parenterally, paracancerally, transmucosally, transdermally, intramuscularly, intravenously, intradermally, subcutaneously, intraperitonealy, intraventricularly, intracranially, or intratumorally.

The composition may comprise a dosage of each active compound (SARM, GnRH agonist or antagonist) in the range of, in one embodiment, 0.1-80 mg/day. In one embodiment the dosage is in the range of 5-50, or in another embodiment, 5-100, or in another embodiment, 5-500 mg/day. In another embodiment, the dosage is in the range of 35-66 mg/day. In another embodiment the dosage is in the range of 40-60 mg/day. In another embodiment the dosage is in a range of 45-60 mg/day. In another embodiment the dosage is in the range of 15-25 mg/day. In another embodiment the dosage is in the range of 55-65 mg/day. In another embodiment the dosage is in the range of 45-60 mg/day. In another embodiment the dosage is in the range of 60-80 mg/day. In another embodiment the dosage is 20 mg/day. In another embodiment the dosage is 40 mg/day In another embodiment the dosage is 60 mg/day. In another embodiment the dosage is 80 mg/day.

In one embodiment, the SARM, or an analog or a metabolite thereof is at a dosage of 20 mg, or in another embodiment, 40 mg, or in another embodiment, 60 mg.

In another embodiment, the composition comprises a suitable carrier or diluent. In one embodiment, the “suitable carrier” or “diluent” is one that is pharmaceutically acceptable carrier, and includes, in other embodiments, any and all solvents, dispersion media, coatings, antibacterial and antifungal agents, isotonic and absorption delaying agents, and the like that are physiologically compatible. Supplementary active compounds can also be incorporated into the compositions. In another embodiment, the carrier or diluent is lactose monohydrate, microcrystalline cellulose, or a mixture thereof.

In another embodiment, the composition may further comprise a lubricant, which in another embodiment, magnesium stearate, or any of a number of such lubricants well known to one skilled in the art. In another embodiment, the composition may further comprise a flow-aid, which, in another embodiment, may be colloidal silicon dioxide. In other embodiments, the composition may further comprise one or more additives such as a binder, a disintegrant, a buffer, a protease inhibitor, a surfactant, a solubilizing agent, a plasticizer, an emulsifier, a stabilizing agent, a viscosity increasing agent, a sweetener, a film forming agent, or a combination thereof.

In another embodiment, the compositions of this invention may be in the form of a pellet, a tablet, a capsule, a solution, a suspension, a dispersion, an emulsion, an elixir, a gel, an ointment, a cream, or a suppository, which may, in other embodiments be in a form suitable for oral, intravenous, intraarterial, intramuscular, subcutaneous, parenteral, transmucosal, transdermal, or topical administration.

In another embodiment, the compositions of this invention may be controlled release compositions, or in another embodiment, immediate release compositions.

In one embodiment, the composition may comprise biodegradable, biocompatible polymers, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

In one embodiment, the composition is a sustained-release formulation, which, in antolier embodiment, comprises a solid ionic complex of an active compound of the invention and a carrier macromolecule, wherein the carrier and compounds used to form the complex are combined at a weight ratio of carrier: actives of for example, 0.5:1 to 0.1:1. In other embodiments, the carrier and actives used to form the complex are combined at a weight ratio of carrier: actives of 0.8:1, 0.7:1, 0.6:1, 0.5:1, 0.4:1, 0.3:1, 0.25:1, 0.2:1, 0.15:1, or 0.1:1. In a preferred embodiment, the complex is not a microcapsule. Ranges intermediate to the above recited values, e.g., 0.8:1 to 0.4:1, 0.6:1 to 0.2:1, or 0.5:1 to 0.1:1 are also intended to be part of this invention. For example, ranges of values using a combination of any of the above-recited values as upper and/or lower limits are intended to be included.

In another embodiment, the concentration of an active compound, or in another embodiment, combined active compounds of the composition is at least 40% by weight, preferably at least 45%, 50%, 55%, 57%, 60%, 65%, 70%, 75%, 80%, 85%, 90%, or 95% by weight. Ranges intermediate to the above recited values, e.g., at least about 50% to about 80%, at least about 60% to about 90%, or at least about 57% to about 80%, are also intended to be part of this invention. For example, ranges of values using a combination of any of the above-recited values as upper and/or lower limits are intended to be included.

In one embodirnent, the composition comprises a therapeutically effective amount of the active compounds of this invention. In one embodiment, the term “therapeutically effective amount” refers to an amount effective, at dosages and for periods of time necessary, to achieve the desired result. A therapeutically effective amount of an LHRH antagonist or agonist and/or a selective androgen receptor modulator may vary according to factors such as the disease state, age, and weight of the individual, and the ability of the LHRH antagonist or agonist and/or the selective androgen receptor modulator (alone or in combination with one or more other drugs) to elicit a desired response in the subject. Dosage regimens may be adjusted to provide the optimum therapeutic response. A therapeutically effective amount is also one in which any toxic or detrimental effects of the LHRH antagonist, agonist and/or the selective androgen receptor modulator are outweighed by the therapeutically beneficial effects.

In one embodiment, the dosage of the LHRH antagonist, agonist and/or the selective androgen receptor modulator is about 10-500 mg/month, about 20-300 mg/month, or about 30-200 mg/month. In a preferred embodiment, the dosage of the LHRH antagonist, agonist and/or the selective androgen receptor modulator is about 30-120 mg/month. Ranges intermediate to the above-recited values, e.g., about 10-200 mg/month, about 30-250 mg/month, or about 100-200 mg/month, are also intended to be part of this invention For example, ranges of values using a combination of any of the above-recited values as upper and/or lower limits are intended to be included. The above-recited dosages may also be calculated and expressed in mg/kg/day. Accordingly, in another embodiment, the dosage of the LHRH antagonist, agonist and/or the selective androgen receptor modulator is about 5-500 μg/kg/day, about 10-400 μg/kg/day, or about 20-200 μg/kg/day. In a preferred embodiment, the dosage of the LHRH antagonist, agonist and/or the selective androgen receptor modulator is about 100 μg/kg/day. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In another embodiment, the present invention is directed to compositions comprising selective androgen receptor modulator compounds, which are antagonist compounds. In another embodiment, the SARMs in the compositions and used in the methods of this invention are agonists, or in another embodiment, behave as agonists in at peripheral tissue sites, and as antagonists in sex organs.

A receptor agonist is a substance, which binds receptors and activates them. A receptor antagonist is a substance, which binds receptors and inactivates them. Thus, in one embodiment, the SARM compounds of the present invention are useful in binding to and activating, or, in another embodiment, inactivating, steroidal hormone receptors. In one embodiment, the antagonist compound of the present invention is an antagonist, which binds the androgen receptor. In another embodiment, the compound has high affinity for the androgen receptor In one embodiment, activation or inactivation of the androgen receptor is a function of its tissue expression.

Assays to determine whether SARMs used in the methods and compositions of this invention are AR agonists or antagonists are well known to a person skilled in the art For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds inhibit the growth of AR containing tissue.

An androgen receptor is an androgen receptor of any species, for example a mammal. In one embodiment, the androgen receptor is an androgen receptor of a human.

The SARM compounds utilized in the compositions and methods of the present invention bind either reversibly or irreversibly to an androgen receptor. In one embodiment, the SARM compounds bind reversibly to an androgen receptor. In another embodiment, the SARM compounds bind reversibly to an androgen receptor of a mammal. In another embodiment, the SARM compounds bind reversibly to an androgen receptor of a human. Reversible binding of a compound to a receptor means that a compound can detach from the receptor after binding.

In another embodiment, the SARM compounds bind irreversibly to an androgen receptor. In one embodiment, the SARM compounds bind irreversibly to an androgen receptor of a mammal. In another embodiment, the SARM compounds bind irreversibly to an androgen receptor of a human. Thus, in one embodiment, the compounds of the present invention may contain a functional group (e.g. affinity label) that allows alkylation of the androgen receptor (i.e. covalent bond formation). Thus, in this case, the compounds are alkylating agents, which bind irreversibly to the receptor and, accordingly, cannot be displaced by a steroid, such as the endogenous ligands DHT and testosterone. An “alkylating agent” is defined herein as an agent, which alkylates (forms a covalent bond) with a cellular component, such as DNA, RNA or enzyme. It is a highly reactive chemical that introduces alkyl radicals into biologically active molecules and thereby prevents their proper functioning. The alkylating moiety is an electrophilic group that interacts with nucleoplilic moieties in cellular components. For example, in one embodiment, an alkylating group is an isocyanate moiety, an electrophilic group, which forms covalent bonds with nucleophilic groups (N, O, S etc.) in cellular components. In another embodiment, an alkylating group is an isothiocyanate moiety, another electrophilic group, which forms covalent bonds with nucleophilic groups (N, O, S etc.) in cellular components. In another embodiment, an alkylating group is a haloalkyl (CH₂X wherein X is halogen), an electrophilic group, which forms covalent bonds with nucleophilic groups in cellular components. In another embodiment, an allcylating group is a haloalkyl-amido (NHCOCH₂X wherein X is halogen), an electrophilic group, which forms covalent bonds with nucleophilic groups in cellular components.

In another embodiment, the compositions of this invention, and methods of use thereof, may comprise, in addition to a SARM and a GnRH agonist or antagonist, another active compound.

In one embodiment, the additional active compound may comprise an anti-androgen, a 5-a reductase inhibitor, an aromatase inhibitor, a SERM, an antiestrogen, or a progestin.

In one embodiment, the 5 alpha reductase inhibitor is MK-906, a product of Merck, Sharp & Dohme (Mc Connell et al., J. Urol. 141: 239A, 1989). In another embodiment, the 5 alpha reductase inhibitor is 17β-N,N-diethylcarbamoyl-4-methyl-4-aza-5.alpha.-androstan-3-one (4-MA) (Brooks et al., Endocrinology 109: 830, 1981; Liang et al., Endocrinology 112: 1460, 1983) In another embodiment, the 5 alpha reductase inhibitor is a 4-azasteroid, which can be formed as in Liang et al., J. Biol. chem. 259: 734-739, 1984; and in Brooks et al., Steroids 47: 1-19, 1986.). In another embodiment, the 5 alpha reductase inhibitor is a 6-methylene-4-pregnene-3,20-dione, for example, as described (Petrow et al., J. Endocrinol. 95: 311-313, 1982). In another embodiment, the 5 alpha reductase inhibitor is a 4-methyl-3-oxo-4-aza-5.alpha.-pregnane-30(s) carboxylate (Kadohama et al., J. Natl. Cancer Inst. 74: 475-486, 1985).

In another embodiment of this invention, the compositions and methods of use thereof may further comprise one or more therapeutic agents. These agents include, but are not limited to: reversible antiandrogens, antiestrogens, anticancer drugs, aromatase inhibitors, progestins, agents acting through other nuclear hormone receptors, selective estrogen receptor modulators (SERM), progesterone, estrogen, PDE5 inhibitors, apomorphine, bisphosphonate, sulfonurea compounds, statins or combinations thereof.

Thus, in one embodiment, the methods of the present invention comprise administering the selective androgen receptor modulator compound and gonadotropin releasing hormone agonist or antagonist, in combination with a 5 alpha reductase inhibitor, or in another embodiment, in combination with a reversible antiandrogen, or in another embodiment, with an antiestrogen, or in another embodiment, with an anticancer drug, or in another embodiment, in combination with an aromatase inhibitor, or in another embodiment, in combination with a progestin, or in another embodiment, in combination with an agent acting through other nuclear hormone receptors, or in another embodiment, in combination with a selective estrogen receptor modulators (SERM), or in another embodiment, in combination with a progesterone, or in another embodiment, in combination with an estrogen, or in another embodiment, in combination with a PDE5 inhibitor, or in another embodiment, in combination with apomorphine, or in another embodiment, in combination with a bisphosphonate.

In one aspect, the invention provides a method for treating or preventing in a subject, a hormone associated condition, by administering to the subject a combination of an LHRH antagonist or agonist and a selective androgen receptor modulator. Administration of a combination of an LHRH antagonist or agonist and a selective androgen receptor modulator can occur prior to the manifestation of symptoms characteristic of the hormone associated condition, such that such a condition is, in one embodiment, prevented or, in another embodiment, delayed in its progression.

It is to be understood that this invention also contemplates simultaneous administration of the SARM and GnRH agonist or antagonist, and that the two need not necessarily be within a single composition. In one embodiment, the SARM is administered concurrently with, or in another embodiment, prior to administering, or in another embodiment following administering a gonadotropin releasing hormone (GnRH) agonist or antagonist.

In one embodiment, administration of either compound may be within a course of 1, or in another embodiment several, or in another embodiment, up to 24 hours of the other. In another embodiment, administration of either compound may be within a course of 1, or in another embodiment several, or in another embodiment, up to 6 days of the other. In another embodiment, administration of either compound may be within a course of 1, or in another embodiment several, or in another embodiment, up to 24 weeks of the other.

In one embodiment, the course of treatment with the SARM and GnRH agonist or antagonist may be for a duration of weeks, months or years, as needed. In one embodiment, treatment may be during symptomatic stages of a given disease or condition, or may be throughout the course of a disease or condition. Alternation in the administration of different SARMs and/or GnRH agonists or antagonists

In one embodiment, the SARM and GnRH agonist or antagonists are delivered via different routes, and, in another embodiment, are in separate compositions.

In one embodiment, the hormone-associated condition treated by the methods of, or with the compositions of this invention, arises as a result of androgen decline, suppression or abrogation. In one embodiment, such decline, suppression or abrogation is a result of the aging process, or in another embodiment, as a result of a pathological condition or disease in a subject. In another embodiment, androgen decline, suppression or abrogation is a by-product of a treatment regimen undergone by a subject, with a particular disease or condition wherein androgen decline, suppression or abrogation is therapeutic.

In one embodiment, the terms “treated”, “treat” or “treating” are synonymous, and include preventative as well as disorder remitative treatment. In another embodiment, the terms “reducing “suppressing” and “inhibiting” have their commonly understood meaning of lessening or decreasing. In another embodiment, the term “progression” means increasing in scope or severity, advancing, growing or becoming worse. In another embodiment, the term “recurrence” means the return of a disease after a remission. In another embodiment, the term “delaying” means stopping, hindering, slowing down, postponing, holding up or setting back.

In another embodiment, the term “administering” refers to bringing a subject in contact with a SARM and GnRH agonist or antagonist, or a composition comprising the same. In one embodiment, administration can be accomplished in vitro, i.e. in a test tube, ex-vivo wherein cells contacted in vitro are reintroduced to a subject, or in vivo, i.e. in cells or tissues of living organisms, for example humans. In one embodiment, the present invention encompasses administering the compounds and compositions of the present invention to a subject.

As used herein, the term “hormone associated condition” includes any disease, disorder, or condition associated with a sex hormone, such as, in one embodiment, testosterone, DHT, estrogen or progesterone. Hormone associated conditions include conditions, diseases, or disorders which affect the organs of the reproductive system. In one embodiment, the organs affected are the prostate, the uterus, ovaries or mammary glands. In another embodiment the hormone associated condition includes conditions, diseases, or disorders which involve an imbalance in the levels of a reproductive-hormone in a subject; and conditions, diseases, or disorders affecting the ability of a subject to reproduce. Examples of hormone-associated conditions may include precocious puberty, prostate cancer, ovarian cancer, benign prostatic hypertrophy, PIN, endometriosis, uterine fibroids, breast cancer, premenstrual syndrome, polycystic ovary syndrome, and diseases, which result from an imbalance, inappropriate expression, or inappropriate regulation of gonadal hormones in humans or animals of either sex.

In one embodiment, the compositions and treatments of a hormone associated disorder may also prove useful for behavior modification (e.g., “chemical castration”), controlling reproduction and other applications as will be known to one skilled in the art.

In one embodiment, the compounds or compositions are used to treat a hormone-associated condition is in a male subject. In one embodiment, the compositions and methods of the present invention are useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia,osteoporosis, benign prostate hyperplasia, alterations in mood and cognition and prostate cancer; c) treatment and/or prevention of acute and/or chronic muscular wasting conditions; d) preventing and/or treating dry eye conditions; e) oral androgen replacement therapy; f) decreasing the incidence of, halting or causing a regression of prostate cancer; g) preventing prostate cancer; h) decreasing the incidence of, preventing, treating, diminishing, halting or causing a regression of precancerous precusors of prostate cancer; i) decreasing the incidence of, preventing, treating, diminishing, halting or causing a regression of benign prostatic hyperplasia; j) or any combination thereof.

In one embodiment, the present invention provides a method of suppressing spermatogenesis in a subject, comprising the step of administering a composition of this invention to the subject, in an amount effective to suppress sperm production, in an amount effective to suppress sperm production.

In another embodiment, the present invention provides a method of contraception in a male subject, comprising the step of administering a composition of this invention to the subject, in an amount effective to suppress sperm production in the subject, thereby effecting contraception in the subject.

In another embodiment, the present invention further provides a method of hormone therapy, comprising the step of administering a composition of this invention to the subject, in an amount effective to effect a change in an androgen-dependent condition.

In another embodiment, the present invention provides a method of hormone replacement therapy comprising administering a composition of this invention to the subject in an amount effective to effect a change in an androgen-dependent condition.

In another embodiment the present invention provides a method of treating, preventing suppressing, inhibiting or reducing the incidence of a condition associated with androgen decline in a male subject, wherein said condition is sexual dysfunction, decreased sexual libido, erectile dysfunction, hypogonadism, sarcopenia, osteopenia, osteoporosis, osteoporosis, alterations in cognition and mood, depression, anemia, hair loss, obesity or muscle wasting said method comprising the step of administering to the subject a composition of this invention.

In another embodiment, the present invention provides a method of treating, preventing, suppressing, inhibiting or reducing the incidence of a condition associated with androgen decline in a male subject, wherein said condition is sexual dysfunction, decreased sexual libido, erectile dysfunction, hypogonadism, sarcopenia, osteopenia, osteoporosis, alterations in cognition and mood, depression, anemia, hair loss, obesity or muscle wasting, said method comprising the step of administering to said subject a selective androgen receptor modulator (SARM) and a gonadotropin releasing hormone (GnRH) agonist or antagonist.

In one embodiment, the term “libido”, means sexual desire, and in another embodiment, the term “erectile”, as used herein, means capable of being erected. An erectile tissue is a tissue, which is capable of being greatly dilated and made rigid by the distension of the numerous blood vessels, which it contains.

In one embodiment, the term “hypogonadism” refers to a condition resulting from or characterised by abnormally decreased functional activity of the gonads, with retardation of growth and sexual development “Osteopenia” refers, in another embodiment to decreased calcification or density of bone. This is a term, which encompasses all skeletal systems in which such a condition is noted.

In another embodiment, the term “osteoporosis” refers to a thinning of the bones with reduction in bone mass due to depletion of calcium and bone protein. Osteoporosis predisposes a person to fractures, which are often slow to heal and heal poorly. Unchecked osteoporosis can lead to changes in posture, physical abnormality, and decreased mobility.

In another embodiment, the term “anemia” refers to the condition of having less than the normal number of red blood cells or less than the normal quantity of hemoglobin in the blood. The oxygen-carrying capacity of the blood is, therefore, decreased. Persons with anemia may feel tired and fatigue easily, appear pale, develop palpitations and become usually short of breath. Anemia is caused by four basic factors: a) hemorrhage (bleeding); b) hemolysis (excessive destruction of red blood cells); c) underproduction of red blood cells; and d) not enough normal hemoglobin. There are many forms of anemia, including aplastic anemia, benzene poisoning, Fanconi anemia, hemolytic disease of the newborn, hereditary spherocytosis, iron deficiency anemia, osteopetrosis, pernicious anemia, sickle cell disease, thalassemia, myelodysplastic syndrome, and a variety of bone marrow diseases. As contemplated herein, the SARM compounds of the present invention are useful in preventing and/or treating any one or more of the above-listed forms of anemia.

In another embodiment, the term “obesity” refers to the state of being well above one's normal weight Traditionally, a person is considered to be obese if they are more than 20 percent over their ideal weight. Obesity has been more precisely defined by the National Institute of Health (NIH) as a Body to Mass Index (BMI) of 30 or above. Obesity is often multifactorial, based on both genetic and behavioral factors. Overweight due to obesity is a significant contributor to health problems. It increases the risk of developing a number of diseases including: Type 2 (adult-onset) diabetes; high blood pressure (hypertension); stroke (cerebrovascular accident or CVA); heart attack (myocardial infarction or MI); heart failure (congestive heart failure); cancer (certain forms such as cancer of the prostate and cancer of the colon and rectum); gallstones and gallbladder disease (cholecystitis); Gout and gouty arthritis; osteoarthritis (degenerative arthritis) of the knees, hips, and the lower back; sleep apnea (failure to breath normally during sleep, lowering blood oxygen); and Pickwickian syndrome (obesity, red face, underventilation and drowsiness). As contemplated herein, the term “obesity” includes any one of the above-listed obesity-related conditions and diseases. Thus the SARM compounds of the present invention are useful in preventing and/or treating obesity and any one or more of the above-listed obesity-related conditions and diseases.

In another embodiment, the present invention provides a method of treating, preventing, suppressing, inhibiting, reducing the incidence of, or reducing relapse of prostate cancer in a male subject, said method comprising the step of administering to the subject a composition of this invention.

In another embodiment, the present invention provides a method of treating, preventing, suppressing, inhibiting, reducing the incidence of, or reducing relapse of prostate cancer in a male subject, said method comprising the step of administering to said subject a selective androgen receptor modulator (SARM) and a gonadotropin releasing hormone (GnRH) agonist or antagonist.

In another embodiment, the term “prostate cancer” refers to one of the most frequently occurring cancers among men, with hundreds of thousands of new cases diagnosed each year. Over sixty percent of newly diagnosed cases of prostate cancer are found to be pathologically advanced, with no cure and a dismal prognosis. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different.

In another embodiment, the present invention further provides a method of treating or preventing benign prostatic hyperplasia, comprising the step of administering to the subject a composition of this invention.

In another embodiment, the present invention further provides a method of treating or preventing benign prostatic hyperplasia, comprising the step of administering to the subject a selective androgen receptor modulator (SARM) and a gonadotropin releasing hormone (GnRH) agonist or antagonist.

In another embodiment, the term “BPH (benign prostate hyperplasia)” is a nonmalignant enlargement of the prostate gland, and is the most common non-malignant proliferative abnormality found in any internal organ and the major cause of morbidity in the adult male. BPH occurs in over 75% of men over 50 years of age, reaching 88% prevalence by the ninth decade. BPH frequently results in a gradual squeezing of the portion of the urethra which traverses the prostate (prostatic urethra). This causes patients to experience a frequent urge to urinate because of incomplete emptying of the bladder and urgency of urination. The obstruction of urinary flow can also lead to a general lack of control over urination, including difficulty initiating urination when desired, as well as difficulty in preventing urinary flow because of the inability to empty urine from the bladder, a condition known as overflow urinary incontinence, which can lead to urinary obstruction and to urinary failure.

In another embodiment, the present invention further provides a method of treating, preventing, suppressing, inhibiting, or reducing the incidence of precancerous precursors of prostate carcionogenesis in a male subject, the method comprising the step of adminstering to the subject administering to the subject a composition of this invention.

In another embodiment, the present invention further provides a method of treating, preventing, suppressing, inhibiting or reducing the incidence of precancerous precursors of prostate cancer in a male subject, said method comprising the step of administering to said subject a selective androgen receptor modulator (SARM) and a gonadotropin releasing hormone (GnRH) agonist or antagonist.

Intermediate endpoint biomarkers are measurable biologic alterations in tissue that occur between the initiation of and the development of frank neoplasia. A biomarker is validated if the final endpoint, cancer incidence, is also reduced by the putative compounds of the present invention. Intermediate biomarkers in cancer may be classified into the following groups: histologic, proliferation differentiation, and biochemical markers In any chemoprevention strategy, the availability of histologically recognizable and accepted precancerous lesions constitutes an important starting point. For the prostate, a histological marker is a precancerous precursor of prostatic adenocarcinoma, of which prostatic intraepithelial neoplasia (PIN) is an example. PIN appears as an abnormal proliferation within the prostatic ducts of premalignant foci of cellular dysplasia and carcinoma in situ without stromal invasion. PIN and histological prostate cancer are morphometrically and phenotypically similar. Thus, the development of high-grade PIN represents an important step in the progression pathway whereby the normal prostate develops PIN, histological prostate cancer, invasive clinical prostate cancer, and metastases. In one embodiment, treating precancerous precursors, such as PIN, and high-grade PIN is another means of preventing prostate carcinogenesis, and represents another method of this invention.

In one embodiment, the subject, for these aspects of the invention, may have an elevated risk of prostate cancer. In another embodiment, the subject may have benign prostatic hyperplasia, prostatic intraepithelial neoplasia (PIN), or an abnormally high level of circulating prostate specific antibody (PSA).

In another embodiment, the invention provides a method of treating, preventing, suppressing, inhibiting or reducing the incidence of a condition associated with androgen decline in a female subject, wherein said condition is sexual dysfunction, decreased sexual libido, hypogonadism, sarcopenia, osteopenia, osteoporosis, alterations in cognition and mood, depression, anemia, hair loss, obesity or muscle wasting, said method comprising the step of administering to said subject to the subject a composition of this invention.

In another embodiment, this invention provides a method of treating, preventing, suppressing, inhibiting or reducing the incidence of a hormone-associated disorder in a female subject, wherein said condition is sexual dysfunction, decreased sexual libido, hypogonadism, sarcopenia, osteopenia, osteoporosis, alterations in cognition and mood, depression, anemia, hair loss, menstrual disorders, infertility, endometriosis, polycystic ovarian syndome, hirsutism, acne, dyslipidemia, hypertension, obesity, muscle wasting, uterine, breast or ovarian cancer, said method comprising the step of administering to said subject a selective androgen receptor modulator (SARM) and a gonadotropin releasing hormone (GnRH) agonist or antagonist.

In another embodiment, the present invention provides a method of suppressing, inhibiting, delaying onset or preventing diabetes, breast cancer, endometrial carcinoma or cardiovascular disease in a female subject sufferring from a hormone-associated disease or disorder, such as polycystic ovarian syndrome. According to this aspect of the invention, and in one embodiment, the method comprises administering a composition of this invention to the subject, or administering to said subject a selective androgen receptor modulator (SARM) and a gonadotropin releasing hormone (GnRH) agonist or antagonist

In another embodiment, the present invention provides a method of treating infertility in a subject in need, the method comprising administering a composition of this invention to the subject, or administering to said subject a selective androgen receptor modulator (SARM) and a gonadotropin releasing hormone (GnRH) agonist or antagonist.

In one embodiment, the methods of the present invention comprise administering at least one gonadotropin releasing hormone agonist or antagonist, its analog, isomer, metabolite, derivative, pharmaceutically acceptable salt, N-oxide, hydrate, acetate or any combination thereof and at least one SARM compound as the active ingredients, however it is to be understood that multiple gonadotropin releasing hormone agonists or antagonists and SARM compounds may be utilized in the methods of this invention, and compositions of the invention, and are to be considered as part of this invention.

It is to be understood that any of the embodiments of this invention, regarding the SARMs, GnRH agonists, antagonists and/or compositions of this invention may represent embodiments for any method described herein, and vice versa, and is to be considered as part of this invention.

The following examples are presented in order to more fully illustrate the preferred embodiments of the invention. They should in no way be construed, however, as limiting the broad scope of the invention.

### Experimental Details Section

### EXAMPLE 1

### Pharmaceutical Compositions

The active ingredient is Formula II (>99.9% pure S-isomer). The inactive ingredients are lactose monohydrate, lactose fast-flo 316, Avicel PH102 (microcrystalline cellulose), magnesium stearate and colloidal silicon dioxide. The blended active and inactive ingredients are filled into white opaque hard gelatin capsules (size one).

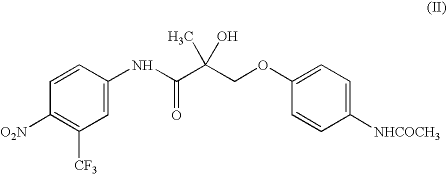

Quantitative Composition

TABLE 1
1 mg FORMULATION

| | | | Weight/Count | | | |
|---|---|---|---|---|---|---|
| | | | Per dosage | | Weight/Count | |
| Ingredient: | Manufacturer: | Excipient Purpose: | unit: | | Per Batch*: | |
| Formula II | ChemSyn | Active | 1.00 | mg | 0.500 | g |
| | Laboratories | | | | | |
| Lactose Monohydrate, | Foremost | Diluent/Filler | 80.00 | mg | 40.000 | g |
| NF (#310 Regular) | | | | | | |
| Lactose Monohydrate, | Foremost | Filler/Flow-Aid | 196.45 | mg | 98.225 | g |
| NF (#316 Fast-Flo | | | | | | |
| Modified, Spray-Dried) | | | | | | |
| Microcrystalline | FMC | Filler/Disintegrant | 30.00 | mg | 15.000 | g |
| Cellulose, NF (Avicel | | | | | | |
| PH102) | | | | | | |
| Silicon Dioxide, | Cabot | Flow-Aid | 1.00 | mg | 0.500 | g |
| Colloidal, USP/NF | | | | | | |
| (Cab-O-Sil M-5P) | | | | | | |
| Magnesium Stearate, | Mallinckrodt | Lubricant | 1.55 | mg | 0.775 | g |
| NF HyQual | | | | | | |
| Capsule, Hard Gelatin | Capsugel | Capsule | 1 | (Count) | 500 | (Count) |
| Size 1, White Opaque | | | | | | |

*Batch size based on 500 capsules but may change depending on requirements

TABLE 2
0.1 mg FORMULATION

| | | | Weight/Count | | | |
|---|---|---|---|---|---|---|
| | | | Per dosage | | Weight/Count | |
| Ingredient: | Manufacturer: | Excipient Purpose: | unit: | | Per Batch*: | |
| Formula II | ChemSyn | Active | 0.10 | mg | 0.050 | g |
| | Laboratories | | | | | |
| Lactose Monohydrate, | Foremost | Diluent/Filler | 80.00 | mg | 40.000 | g |
| NF (#310 Regular) | | | | | | |
| Lactose Monohydrate, | Foremost | Filler/Flow-Aid | 197.35 | mg | 98.675 | g |
| NF (#316 Fast-Flo | | | | | | |
| Modified, Spray-Dried) | | | | | | |
| Microcrystalline | FMC | Filler/Disintegrant | 30.00 | mg | 15.000 | g |
| Cellulose, NF (Avicel | | | | | | |
| PH102) | | | | | | |
| Silicon Dioxide, | Cabot | Flow-Aid | 1.00 | mg | 0.500 | g |
| Colloidal, USP/NF | | | | | | |
| (Cab-O-Sil M-5P) | | | | | | |
| Magnesium Stearate, | Mallinckrodt | Lubricant | 1.55 | mg | 0.775 | g |
| NF HyQual | | | | | | |
| Capsule, Hard Gelatin | Capsugel | Capsule | 1 | (Count) | 500 | (Count) |
| Size 1, White Opaque | | | | | | |

*Batch size based on 500 capsules but may change depending on requirements

Specifications and Analytical Methods for Inactive Compounds

All inactive ingredients included in the formulation have monographs that denote full conipendial testing per Standard Operating Procedure of Metrics, Inc. (1240 Sugg Parkway Greenville, N.C. 27834) with the exception of the active ingredient and the capsule shell. Active pharmaceutical ingredient and capsule shells are provided with Certificate of Analysis.

Method of Manufacturing

Capsules of Formula II are manufactured using the formulations as set forth in Table 1 (1 mg formulation) and Table 2 (0.1 mg formulation).

For 1 mg Formula II capsules: the indicated amount of active and inactive ingredients are dispensed. 0.5 grams of Formula II (active pharmaceutical ingredient, API) are diluted by placing API and an equal part of lactose monohydrate (0.5 grams) in mortar. The mixture is ground with a pestle until homogenous. The mixture is diluted again by adding one additional gram of lactose monohydrate to the mixture and grinding until homogenous. The diluted active:lactose monohydrate mixture is blended with 38.5 grams of lactose monohydrate, 98.225 grams of lactose fast-flo, and 15 grams of Avicel PH102 in a one pint V-shell blender for 15 minutes. Approximately 10 grams of the blend is removed and added to 0.5 grams of Cab-O-Sil. The mixture is mixed with a spatula and screened through a 20-mesh screen. 0.775 grams of magnesium stearate are independently screened through a 20-mesh screen. The screened ingredients (10 grams of initial blend with Cab-O-Sil, and magnesium stearate) are added to the remainder of the initial blend in the one pint V-shell blender. All ingredients are blended together in a V-shell blender for five minutes. Capsule shells (500 count) are dispensed into a Chemipharm, Manual Capsule Filler. 31 grams of blended mixture are manually filled into 100 capsules using the Chemipharm Manual Capsule Filler. The capsules are manually packaged and labeled. Each capsule contains 1 milligram of active and 309 milligrams of inactive ingredients.

For 0.1 mg Formula II capsules: the same Method of Manufacturing is used, the amounts of Formula II API and inactive ingredients are adjusted accordingly (Table 2).

### EXAMPLE 2

### Additional Nonsteroidal Ligands With Androgenic and Anabolic Activity Synthetic Procedures

(2R)-1-Methacryloylpyrrolidin-2-carboxylic Acid (R-129). D-Proline (R-128, 14.93 g, 0.13 mol) was dissolved in 71 mL of 2 N NaOH and cooled in an ice bath; the resulting alkaline solution was diluted with acetone (71 mL). An acetone solution (71 mL) of metacryloly chloride 127 (13.56 g, 0.13 mol) and 2N NaOH solution (71 mL) were simultaneously added over 40 min to the aqueous solution of D-proline in an ice bath. The pH of the mixture was kept at 10-11° C. during the addition of the metacryloly chloride. After stirring (3 h, room temperature), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite, and evaporated in vacuo to give the crude product as a colorless oil. Recrystallization of the oil from ethyl ether and hexanes afforded 16.2 (68%) of the desired compound as colorless crystals: mp 102-103° C. (lit. [214] mp 102.5-103.5° C.); the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral canter), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00; H, 7.15; N 7.65. Found: C, 59,13; H, 7.19; N, 7.61.

(3R,8aR)-3-Bromomethyl-3-methyl-tetrahydro-pyrrolo[2,1c] [1,4]oxazinc-1,4-dione (R, R-130). A solution of NBS (23.5 g, 0.132 mol) in 100 mL of DMF was added dropwise to a stirred solution of compound R-129 (16.1 g, 88 mmol) in 70 mL of DMF under argon at room temperature, and the resulting mixture was stirred 3 days. The solvent was removed in vacuo, and a yellow solid was precipitated. The solid was suspended in water, stirred overnight at room temperature, filtered, and dried to give 18.6 (81%) (smaller weight when dried ˜34%) of the title compound as a yellow solid: mp 152-154° C. (lit. [214] mp 107-109° C. for the S-isomer); ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24; H, 4.61; N, 5.34. Found: C, 41.46; H, 4.64; N, 5.32.

(2R)-3-Bromo-2-hydroxy-2-methytpropanoic Acid (R-131). A mixture of bromolactone R-130 (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1. which, in turn, was extracted with ethyl acetate (100 mL×4). The combined organic solution was dried over Na₂SO₄, filtered through Celite, and evaporated in vacuo to dryness Recrystallization from toluene afforded 10.2 g (86%) of the desired compound as colorless crystals: mp 107-109° C. (lit. [214] mp 109-113° C. for the S-isomer); ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5° (c=2.6, MeOH); Anal. Calcd for C₄H₇BrO₃: C, 26.25; H, 3.86. Found: C, 26.28; H, 3.75.

N-[4-Nitro-3-(trifluoromethylphenyl]-(2R)-3-bromo-2-hydroxy-2-methylpropanamide (R-132). Thionyl chloride (8.6 g, 72 mmol) was added dropwise under argon to a solution of bromoacid R-131 (11.0 g, 60 mmol) in 70 mL of DMA at −5 to −10° C. The resulting mixture was stirred for 2 h under the same conditions. A solution of 4-nitro-3-trifluoromethyl-aniline (12.4 g, 60 mmol) in 80 mL of DMA was added dropwise to the above solution, and the resulting mixture was stirred overnight at room temperature. The solvent was removed on Rotavapor using high vacuum oil pump; the residue was diluted with saturated NaHCO₃ solution, and extracted with ethyl ether (100 mL×3). Combined extracts were dried over anhydrous Na₂SO₄, filtered through Celite, and purified by flash chromatography on silica gel, using methylene chloride as eluent to afford 18.0 g (80%) of the desired compound: mp 98-100° C. (R_{f}=0.2, silica gel, CH₂Cl₂); ¹H NMR (300 MHz, DMSO-d₆) δ 10.54 (s, 1H, NH), 8.54 (d, J=2.1 Hz, 1H, ArH), 8.34 (dd, J=9.0 Hz, J=2.1 Hz, 1H, ArH), 8.18 (d, J=9.0 Hz, 1H, ArH), 6.37 (s, 1H, OH), 3.82 (d, J=10.4 Hz, 1H, CHHₐ), 3.58 (d, J=10.4 Hz, 1H, CHH_{b}), 1.48 (s, 3H, Me); ¹³C NMR (75 MHz, DMSO-d₆) δ 173.6 (C═O), 143.0, 127.2, 123.2, 122.6 (q, J=33.0 Hz), 122.0 (q, J=271.5 Hz), 118.3 (q, J=6.0 Hz), 74.4, 41.4, 24.9; IR (KBr) 3344 (OH), 1680 (C═O), 1599, 1548 (C═C, Ar), 1427, 1363, 1161 cm⁻¹; MS (ESI): m/z 370.8 (M)⁺; Anal. Calcd. for C₁₁H₁₀BrN₂O₄: C, 35.60; H, 2.72; N, 7.55. Found: C, 35.68; H, 2.72; N, 7.49.

N-[4-nitro-3-trifluoromethyl)phenyl]-(2S)-3-[4-(acetylamino)phenoxy]-2-hydroxy-2-methylpropanamide (S-147, Compound IV). The title compound was prepared from compound R-132 (0.37 g, 1.0 mmol), 4-acetamidophenol (0.23 g, 1.5 mmol) K₂CO₃ (0.28 g, 2.0 mmol), and 10% of benzyltributylaunmonium chloride as a phase transfer catalyst in 20 mL of methyl ethyl ketone was heated at reflux overnight under argon. The reaction was followed by TLC, the resulting mixture was filtered through Celite, and concentrated in vacuo to dryness. Purification by flash column clromatography on silica gel (hexanes-ethyl acetate, 3:1) yielded 0.38 g (86%) (R_{f}=0.18 hexanes-ethyl acetate, 3:1) of the desired compound as a light yellow powder: mp 70-74° C.; The solid can be recrystalized from ethyl acetate and hexane); ¹H NMR (300 MHz, DMSO-d₆) δ 10.62 (s, 1H, NH), 9.75 (s, 1H, NH), 8.56 (d, J=1.9 Hz, 1H, ArH), 8.36 (dd, J=9.1 Hz, J=1.9 Hz, 1H, ArH), 8.18 (d, J=9.1 Hz, 1H, ArH), 7.45-7.42 (m, 2H, ArH), 6.85-6.82 (m, 2H, ArH), 6.25 (s, 1H, OH), 4.17 (d, J=9.5 Hz, 1H, CHHₐ), 3.94 (d, J=9.5 Hz, 1H, CHH_{b}), 1.98 (s, 3H, Me), 1.43 (s, 3H, Me); ¹³C NMR (75 MHz, DMSO-d₆) δ 174.6 (C═O), 167.7, 154.2, 143.3, 141.6, 132.8, 127.4, 123.0, 122.7 (q, J=33.0 Hz), 122.1 (q, J=271.5 Hz), 120.1, 118.3 (q, J=6.0 Hz), 114.6, 74.9, 73.8, 23.8, 23.0; IR (KBr) 3364 (OH), 1668 (C═O), 1599, 1512 (C═C, Ar), 1457, 1415, 1351, 1323, 1239, 1150 1046 cm⁻¹; MS (ESI): m/z 464.1 (M+Na)⁺; Anal. Calcd. for C₁₉H₁₈F₃N₃O₆: C, 51.71; H, 4.11; N, 9.52. Found: C, 52.33; H, 4.40; N, 9.01.

The synthesis of the various-ether analogs of Compound IV, such as, but not limited to, compounds I-III and V-VII provided herein, utilizes the common intermediate that is the final reaction step. Bromo-intermediates are used winch allow various phenolic compounds to displace the bromide to give the desired ether product. Bromohydrin was converted to an epoxide and to open the epoxide to give the same desired ether product.

### EXAMPLE 3

### GnRH Agonist and SARM Compositions

A Tablet formulation, with scored tablets for oral use, may be prepared containing, in one embodiment, 500 mg. of each active ingredient. The tablets may be prepared, in one embodiment, from the following ingredients:

| | Gm. |
|---|---|
| Leuprorelin | 5000 |
| Formula II | 5000 |
| Starch, U.S.P. | 350 |
| Talc, U.S.P. | 250 |
| Calcium stearate | 35 |

The active ingredients are granulated with a 4% w./v. aqueous solution of methylcellulose U.S.P. (1500 cps). Tablets containing 0.1, 1, 5, 10, 15, 25, 50, and 100 mg. of each active ingredient may also be prepared, in other embodiments, by substituting 1, 10, 50, 100, 150, 250, 500, and 1000 gm. of 2500 gm. in the above formulation. To the dried granules is added a mixture of the remainder of the ingredients and the final mixture compressed into, tablets of proper weight.

Capsules—hard gelatin capsules for oral use, each containing 250 mg. of active ingredients may be prepared, in another embodiment from the following ingredients:

| | Gm |
|---|---|
| Leuprorelin | 2500 |
| Formula II | 2500 |
| Lactose, U.S.P. | 1000 |
| Starch, U.S.P. | 300 |
| Talc, U.S.P. | 65 |
| Calcium Stearate | 25 |

The active ingredients are mixed with the starch lactose mixture followed by the talc and calcium stearate. The final mixture is then encapsulated in the usual manner. Capsules containing 0.1, 1, 5, 10, 15, 25, 50, and 100 mg. of each active ingredient is also prepared by substituting 1, 10, 50, 100, 150, 250, 500, and 1000 gm. of 2500 gm. in the above formulation. In another embodiment, the concentration of the SARM is 10, or in another embodiment 25, or in another embodiment 50 % that of the GnRH agonist, in any composition of this invention.

Soft elastic capsules—One-piece soft elastic capsules for oral use, each containing 500 mg. of each, or 250 mg of each active material are prepared in the usual manner by first dispersing the active material in sufficient corn oil to render the material capsulatable.

Aqueous suspension—An aqueous suspension for oral use containing in each 5 ml., 0.25 g. of each active ingredient is prepared from the following ingredients:

| | Gm. |
|---|---|
| Leuprorelin | 500 |
| Formula II | 500 |
| Methylparaben, U.S.P. | 7.5 |
| Propylparaben, U.S.P. | 2.5 |
| Saccharin sodium | 12.5 |
| Glycerin | 3000 |
| Tragacanth powder | 10 |
| Orange oil flavor | 10 |
| F.D. & C. orange dye | 7.5 |
| Deionized water, q.s. to 10,000 ml | |

### EXAMPLE 4

### GnRH Antagonist and SARM Compositions

A Tablet formulation, with scored tablets for oral use, may be prepared containing, in one embodiment, 500 mg. of each active ingredient. The tablets may be prepared, in one embodiment, from the following ingredients:

| | Gm. |
|---|---|
| Cetrorelix | 5000 |
| Formula II | 5000 |
| Starch, U.S.P. | 350 |
| Talc, U.S.P. | 250 |
| Calcium stearate | 35 |

The active ingredients are granulated with a 4% w./v. aqueous solution of methylcellulose U.S.P. (1500 cps). Tablets containing 0.1, 1, 5, 10, 15, 25, 50, and 100 mg. of each active ingredient may also be prepared, in other embodiments, by substituting 1, 10, 50, 100, 150, 250, 500, and 1000 gm. of 2500 gm. in the above formulation. To the dried granules is added a mixture of the remainder of the ingredients and the final mixture compressed into tablets of proper weight.

Capsules—hard gelatin capsules for oral use, each containing 250 mg. of active ingredients may be prepared in another embodiment from the following ingredients:

| | Gm |
|---|---|
| Cetrorelix | 2500 |
| Formula II | 2500 |
| Lactose, U.S.P. | 1000 |
| Starch, U.S.P. | 300 |
| Talc, U.S.P. | 65 |
| Calcium Stearate | 25 |

The active ingredients are mixed with the starch lactose mixture followed by the talc and calcium stearate. The final mixture is then encapsulated in the usual manner. Capsules containing 0.1, 1, 5, 10, 15, 25, 50, and 100 mg. of each active ingredient is also prepared by substituting 1, 10, 50, 100, 150, 250, 500, and 1000 gm. of 2500 gm. in the above formulation. In another embodiment, the concentration of the SARM is 10, or in another embodiment 25, or in another embodiment 50% that of the GnRH agonist, in any composition of this invention.

Soft elastic capsules—One-piece soft elastic capsules for oral use, each containing 500 mg. of each, or 250 mg of each active material are prepared in the usual manner by first dispersing the active material in sufficient corn oil to render the material capsulatable.

Aqueous suspension—An aqueous suspension for oral use containing in each 5 ml., 0.25 g. of each active ingredient is prepared from the following ingredients:

| | Gm. |
|---|---|
| Cetrorelix | 500 |
| Formula II | 500 |
| Methylparaben, U.S.P. | 7.5 |
| Propylparaben, U.S.P. | 2.5 |
| Saccharin sodium | 12.5 |
| Glycerin | 3000 |
| Tragacanth powder | 10 |
| Orange oil flavor | 10 |
| F.D. & C. orange dye | 7.5 |
| Deionized water, q.s. to 10,000 ml | |

### EXAMPLE 5

### Treatment of Prostate Cancer With GnRH Antagonist and SARM Compositions

Randomized, parallel-group, studies are conducted in adult male patients with prostate cancer, including patients with local or regional disease, patients with metastatic disease (stage D1 or D2); patients with rising prostate specific antigen (PSA) levels after radical prostatectomy, radiation therapy, or other local therapy; and patients scheduled for their initial course of intermittent therapy are entered into the study.

Patients are randomized to receive, in one embodiment abarelix depot 100 mg and formula II, by intramuscular (IM) injection on days 1, 29, 57, 85, 113, and 141. As clinically indicated, patients may continue treatment with study drugs for up to 1 year with injections on day 169 and every 28 days thereafter (up to 7 additional injections beyond day 141).

Depending on their day 169 testosterone levels (i.e., if >50 ng/dL), patients may receive an extra injection of study drug on day 183 (2 weeks before their next regularly scheduled injection).

Median follicle-stimulating hormone (FSH) level is measured over the course of treatment and compared to baseline, with FSH suppression serving as an indicator for response to therapy.

It will be appreciated by a person skilled in the art that the present invention is not limited by what has been particularly shown and described hereinabove. Rather, the scope of the invention is defined by the claims that follow:

1. A composition comprising a gonadotropin releasing hormone agonist, its analog, isomer, metabolite, derivative, pharmaceutically acceptable salt, N-oxide, hydrate, acetate or a combination thereof; and a selective androgen receptor modulator (SARM) compound having in-vivo androgenic and anabolic activity of a nonsteroidal ligand for the androgen receptor, said compound represented by the structure of formula I: 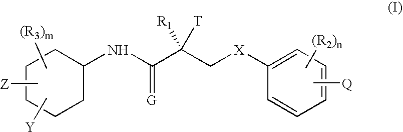
wherein:
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃,
CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 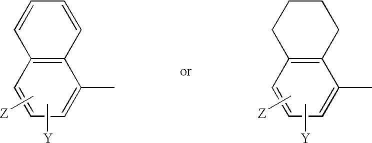
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

2-10. (canceled)

11. The composition according to claim 1, wherein G is O, T is OH, R1 is CH₃, X is O, Z is NO₂, Y is CF₃, and Q is NCS.

12-24. (canceled)

25. A composition comprising a gonadotropin releasing hormone agonist, its analog, isomer, metabolite, derivative, pharmaceutically acceptable salt, N-oxide, hydrate, acetate or a combination thereof; and a selective androgen receptor modulator (SARM) compound having in-vivo androgenic and anabolic activity of a nonsteroidal ligand for the androgen receptor, said compound represented by the structure of formula II: 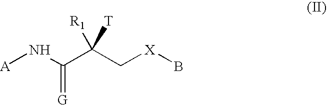
Wherein:
X is a bond, O, CH2, NH, S, Se, PR, NO or NR;
G is O or S;
R1 is CH3, CH2F, CHF2, CF3, CH2CH3, or CF2CF3;
T is OH, OR, —NHCOCH3, or NHCOR;
R is alkyl, haloalkyl, dihaloalkyl, trihaloalkyl, CH2F, CHF2, CF3, CF2CF3, aryl, phenyl, halogen, alkenyl or OH;
A is a ring selected from: 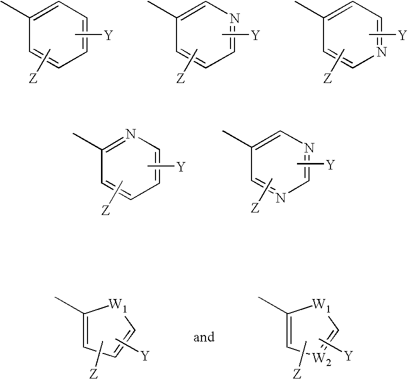
B is a ring selected from: 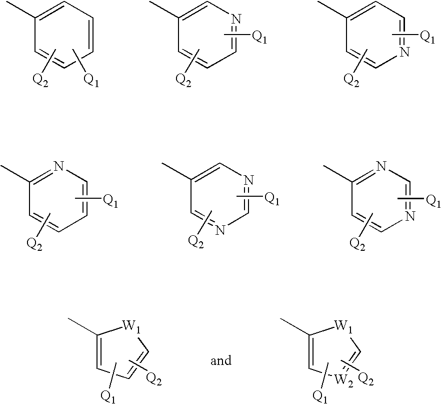
wherein A and B cannot simultaneously be a benzene ring;
Z is NO2, CN, COOH, COR, NHCOR or CONHR;
Y is CF3, F, I, Br, Cl, CN CR3 or SnR3;
Q1 is NCS, SCN, NCO or OCN;
Q2 is a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCP3, NHCSR NHSO2CH3, NHSO2R, OR, COR, OCOR, OSO2R, SO2R, SR, 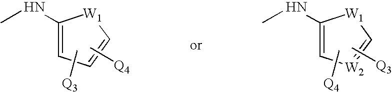
Q3 and Q4 are independently of each other a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R or SR;
W1 is O NH, NR, NO or S;
W2 is N or NO and
wherein all unspecified positions can be substituted or unsubstituted.

26-33. (canceled)

34. The composition according to claim 25, wherein G is O, T is OH, R1 is CH₃, X is O, Z is NO₂, Y is CF₃, and Q1 is NCS.

35-46. (canceled)

47. A composition comprising a gonadotropin releasing hormone agonist, its analog, isomer, metabolite, derivative, pharmaceutically acceptable salt, N-oxide, hydrate, acetate or a combination thereof; and a selective androgen receptor modulator (SARM) compound having in-vivo androgenic and anabolic activity of a nonsteroidal ligand for the androgen receptor, said compound represented by the structure of formula III: 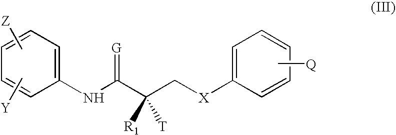
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is NHCOCH₃, SCN, NCS, OCN, or NCO;
R is alk-yl, haloalkyl, dihaloalkyl, trihaloalkyl, CH₂F, CHF₂, CF₃,
CF₂CF₃, aryl, phenyl, halogen, alkenyl or OH; and
R₁ is CH₃, CH₂F, CHF₂, CF₃, CH₂CH₃, or CF₂CF₃.

48.-55. (canceled)

56. The composition according to claim 47, wherein G is O, T is OH, R is CH₃, X is O, Z is NO₂, Y is CF₃, and Q is NCS.

57-58. (canceled)

59. The composition according to claim 47, represented by the structure of formula IV:

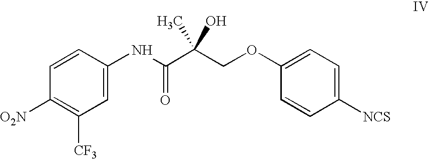

60-139. (canceled)

140. A method of treating, preventing, suppressing, inhibiting or reducing the incidence of a condition associated with androgen decline in a male subject, wherein said condition is sexual dysfunction, decreased sexual libido, erectile dysfunction, hypogonadism, sarcopenia, osteopenia, osteoporosis, alterations in cognition and mood, depression, anemia, hair loss, obesity or muscle wasting, said method comprising the step of administering to said subject a selective androgen receptor modulator (SARM) and a gonadotropin releasing hormone (GnRH) agonist or antagonist.

141. The method of claim 140, wherein said selective androgen receptor modulator (SARM) is represented by the structure of formula I: 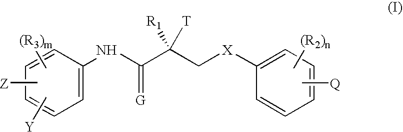
wherein:
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached foris a fused ring system represented by the structure: 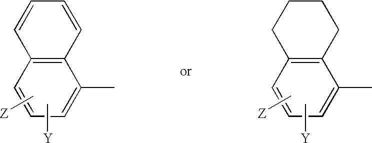
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

142-149. (canceled)

150. The method of claim 140, wherein G is O, T is OH, R1 is CH₃, X is O, Z is NO₂, Y is CF₃, and Q is NCS.

151. The method of claim 140, wherein said selective androgen receptor modulator (SARM) is represented by the structure of formula II: 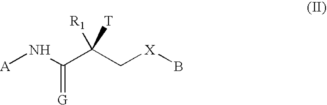
Wherein:
X is a bond, O, CH2, NH, S, Se, PR, NO or NR;
G is O or S;
R1 is CH3, CH2F, CHF2, CF3, CH2CH3, or CF2CF3;
T is OH, OR, —NHCOCH3, or NHCOR;
R is alkyl, haloalkyl, dihaloalkyl, trihaloalkyl, CH2F, CHF2, CF3, CF2CF3, aryl, phenyl, halogen, alkenyl or OH;
A is a ring selected from: 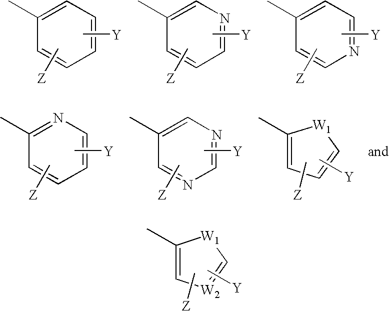
B is a ring selected from: 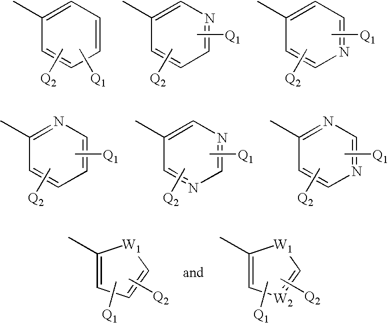
wherein A and B cannot simultaneously be a benzene ring;
Z is NO2, CN, COOH, COR, NHCOR or CONHR;
Y is CF3, F, I, Br, Cl, CN CR3 or SnR3;
Q1 is NCS, SCN, NCO or OCN;
Q2 is a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R, SR, 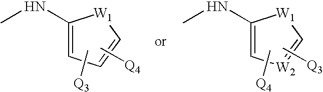
Q3 and Q4 are independently of each other a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R or SR;
W1 is O, NH, NR, NO or S;
W2 is N or NO and
wherein all unspecified positions can be substituted or unsubstituted.

152-157. (canceled)

160. The method of claim 151, wherein G is O, T is OH, R1 is CH₃, X is O, Z is NO₂, Y is CF₃, and Q1 is NCS.

161. The method of claim 140, wherein said selective androgen receptor modulator (SARM) is represented by the structure of formula III: 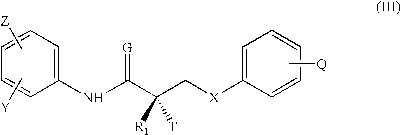
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR,
Y is CF₃, F, I, Br, Cl, CN, CR₃ or SnR₃;
Q is NHCOCH₃, SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

162-169. (canceled)

170. The method of claim 161, wherein G is O, T is OH, R₁ is CH₃, X is O, Z is NO₂, Y is CF₃, and Q is NCS.

171-176. (canceled)

177. A method of treating, preventing, suppressing, inhibiting, reducing the incidence of, or reducing relapse of prostate cancer in a male subject, said method comprising the step of administering to said subject a selective androgen receptor modulator (SARM) and a gonadotropin releasing hormone (GnRH) agonist or antagonist.

178. The method of claim 177, wherein said selective androgen receptor modulator (SARM) is represented by the structure of formula I: 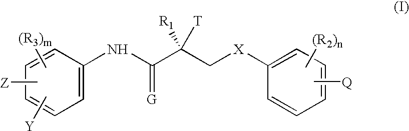
wherein:
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 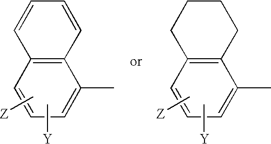
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

179-186. (canceled)

187. The method of claim 178, wherein G is O, T is OH, R1 is CH₃, X is O, Z is NO₂, Y is CF₃, and Q is NCS.

188. The method of claim 177, wherein said selective androgen receptor modulator (SARM) is represented by the structure of formula II: 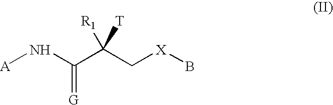
Wherein:
X is a bond, O, CH₂, NH, S, Se, PR, NO or NR;
G is O or S;
R1 is CH3, CH2F, CHF2, CF3, CH2CH3, or CF2CF3;
T is OH, OR, —NHCOCH3, or NHCOR;
R is alkyl, haloalkyl, dihaloalkyl, trihaloalkyl, CH2F, CHF2, CF3, CF2CF3, aryl, phenyl, halogen, alkenyl or OH;
A is a ring selected from: 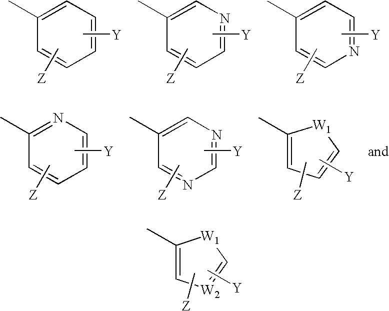
B is a ring selected from: 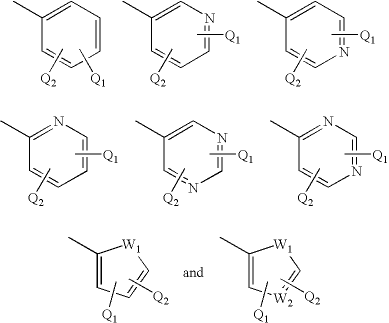
wherein A and B cannot simultaneously be a benzene ring;
Z is NO2, CN, COOH, COR, NHCOR or CONHR;
Y is CF3, F, I, Br, Cl, CN CR3 or SnR3,
Q1 is NCS, SCN, NCO or OCN;
Q2 is a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R, SR, 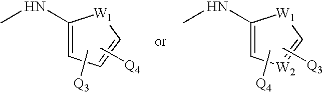
Q3 and Q4 are independently of each other a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R or SR;
W1 is O, NH, NR, NO or S;
W2 is N or NO and
wherein all unspecified positions can be substituted or unsubstituted .

189-196. (canceled)

197. The method of claim 188, wherein G is O, T is OH, R1 is CH₃, X is O, Z is NO₂, Y is CF₃, and Q1 is NCS.

198. The method of claim 177, wherein said selective androgen receptor modulator (SARM) is represented by the structure of formula III: 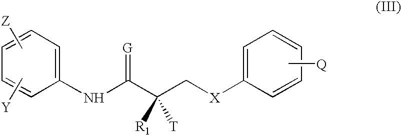
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is NHCOCH₃, SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

199.-206. (canceled)

207. The method of claim 198, wherein G is O, T is OH, R₁ is CH₃, X is O, Z is NO₂, Y is CF₃, and Q is NCS.

208-213. (canceled)

214. A method of treating, preventing, suppressing, inhibiting or reducing the incidence of precancerous precursors of prostate cancer in a male subject, said method comprising the step of adrninistering to said subject a selective androgen receptor modulator (SARM) and a gonadotropin releasing hormone (GnRH) agonist or antagonist.

215. The method of claim 214, wherein said selective androgen receptor modulator (SARM) is represented by the structure of formula I: 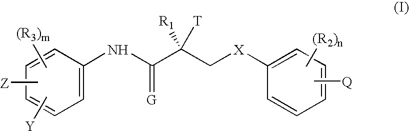
wherein:
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, aLkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 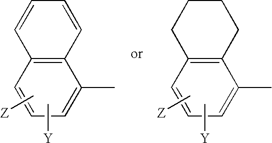
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

216-223. (canceled)

224. The method of claim 215, wherein G is O, T is OH, R1 is CH₃, X is O, Z is NO₂, Y is CF₃, and Q is NCS.

225. The method of claim 214 wherein said selective androgen receptor modulator (SARM) is represented by the structure of formula II: 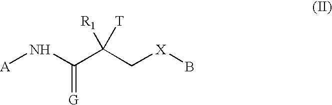
Wherein:
X is a bond, O, CH2, NH, S, Se, PR, NO or NR;
G is O or S;
R1 is CH3, CH2F, CHF2, CF3, CH2CH3, or CF2CF3;
T is OH, OR, —NHCOCH3, or NHCOR;
R is alkyl, haloalkyl, dihaloalkyl, trihaloalkyl, CH2F, CHF2, CF3, CF2CF3, aryl, phenyl, halogen, alkenyl or OH;
A is a ring selected from: 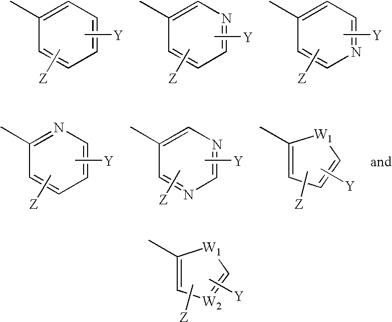
B is a ring selected from: 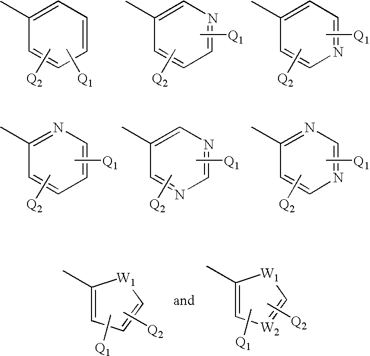
wherein A and B cannot simultaneously be a benzene ring;
Z is NO2, CN, COOH, COR, NHCOR or CONHR;
Y is CF3, F, I, Br, Cl, CN CR3 or SnR3;
Q1 is NCS, SCN, NCO or OCN;
Q2 is a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R, SR, 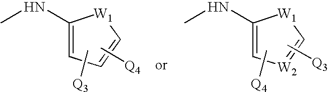
Q3 and Q4 are independently of each other a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R or SR;
W1 is O, NH, NR, NO or S;
W2 is N or NO and
wherein all unspecified positions can be substituted or unsubstituted.

226-234. (canceled)

235. The method of claim 225, wherein G is O, T is OH, R1 is CH₃, X is O, Z is NO₂, Y is CF₃, and Q1 is NCS.

236. The method of claim 214, wherein said selective androgen receptor modulator (SARM) is represented by the structure of formula III: 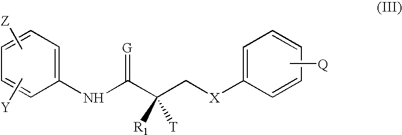
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is NHCOCH₃, SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CBF₂, CF₃, CH₂CH₃, or CF₂CF₃.

237-243. (canceled)

244. The method of claim 235, wherein G is O, T is OH, R₁ is CH₃, X is O, Z is NO₂, Y is CF₃, and Q is NCS.

245-250. (canceled)

251. A method of treating, preventing, suppressing, inhibiting or reducing the incidence of a hormone-associated disorder in a female subject, wherein said condition is sexual dysfunction, decreased sexual libido, hypogonadism, sarcopenia, osteopenia, osteoporosis, alterations in cognition and mood, depression, anemia, hair loss, menstrual disorders, infertility, endometriosis, polycystic ovarian syndome, hirsutism, acne, dyslipidemia, hypertension, obesity, muscle wasting, breast cancer, uterine cancer or ovarian cancer said method comprising the step of administering to said subject a selective androgen receptor modulator (SARM) and a gonadotropin releasing hormone (GnRH) agonist or antagonist.

252. The method of claim 251, wherein said selective androgen receptor modulator (SARM) is represented by the structure of formula I: 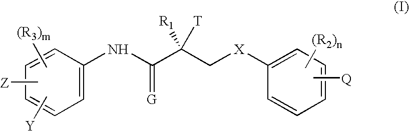
wherein:
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fuised ring system represented by the structure: 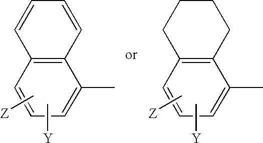
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

253-260. (canceled)

261. The method of claim 252, wherein G is O, T is OH, R1 is CH₃, X is O, Z isNO₂, Y is CF₃, and Q is NCS.

262. The method of claim 251, wherein said selective androgen receptor modulator (SARM) is represented by the structure of formula II: 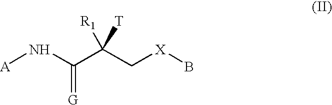
Wherein:
X is a bond, O, CH2, NH, S, Se, PR, NO or NR;
G is O or S;
R1 is CH3, CH2F, CHF2, CF3, CH2CH3, or CF2CF3;
T is OH, OR, —NHCOCH3, or NHCOR;
R is alkyl, haloalkyl, dihaloalkyl, trihaloalkyl, CH2F, CHF2, CF3, CF2CF3, aryl, phenyl, halogen, alkenyl or OH;
A is a ring selected from: 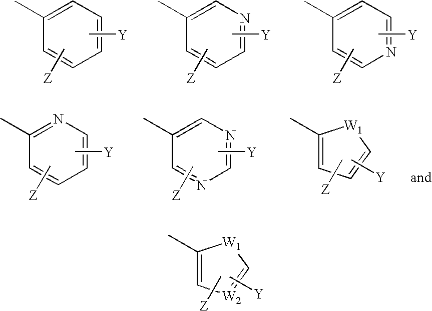
B is a ring selected from: 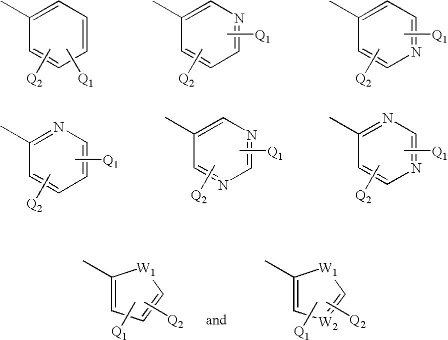
wherein A and B cannot simultaneously be a benzene ring;
Z is NO2, CN, COOH, COR, NHCOR or CONHR;
Y is CF3, F, I, Br, Cl, CN CR3 or SnR3;
Q1 is NCS, SCN, NCO or OCN;
Q2 is a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R, SR, 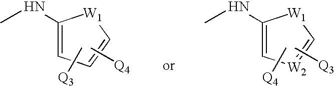
Q3 and Q4 are independently of each other a hydrogen, alkyl, halogen, CF3, CN CR3, SnR3, NR2, NHCOCH3, NHCOCF3, NHCOR, NHCONHR, NHCOOR, OCONHR, CONHR, NHCSCH3, NHCSCF3, NHCSR NHSO2CH3, NHSO2R, OR, COR, OCOR, OSO2R, SO2R or SR;
W1 is O, NH, NR, NO or S;
W2 is N or NO and
wherein all unspecified positions can be substituted or unsubstituted.

263-270. (canceled)

271. The method of claim 262, wherein G is O, T is OH, R1 is CH₃, X is O, Z is NO₂, Y is CF₃, and Q1 is NCS.

272. The method of claim 251, wherein said selective androgen receptor modulator (SARM) is represented by the structure of formula III: 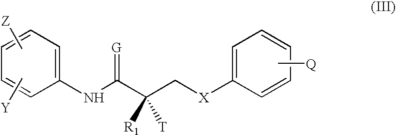
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is NHCOCH₃, SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

273.-280. (canceled)

281. The method of claim 272, wherein G is O, T is OH, R₁ is CH₃, X is O, Z is NO₂, Y is CF₃, and Q is NCS.

282-286. (canceled)
